(19) United States
(12) Patent Application Publication
(10) Pub. No.: US 2012/0316337 A1
(43) Pub. Date: Dec. 13, 2012

(54) METHOD FOR PREPARING CHEMICAL COMPOUNDS OF INTEREST BY NUCLEOPHILIC AROMATIC SUBSTITUTION OF AROMATIC CARBOXYLIC ACID DERIVATIVES SUPPORTING AT LEAST ONE ELECTRO-ATTRACTIVE GROUP

(75) Inventors: Jacques Mortier, Ecommoy (FR); Anne-Sophie Castanet, Le Mans (FR); Mickael Belaud-Rotureau, Le Mans (FR)

(73) Assignees: UNIVERSITE DU MAINE, Le Mans (FR); CENTRE NATIONAL DE LA RECHERCHE SCIENTIFIQUE, Paris (FR)

(21) Appl. No.: 13/578,673

(22) PCT Filed: Feb. 17, 2011
(86) PCT No.: PCT/FR2011/050337

§ 371 Date: Aug. 13, 2012

(30) Foreign Application Priority Data
Feb. 19, 2010 (FR) 1051226
Dec. 10, 2010 (FR) PCT/FR2010/052674

(51) Int. Cl. C07C 227/08; C07C 51/353; C07D 237/30; C07D 215/48; C07D 217/00; C07D 295/155; C07C 51/367

(52) U.S. Cl. 544/237; 562/458; 544/395; 562/453; 562/457; 562/490; 562/467; 546/170; 546/147

(57) ABSTRACT

Method for preparing carboxylic acid derivatives by aromatic nucleophilic substitution, in which a carboxylic acid derivative having a single carboxyl functional group, or one of the salts thereof, the carboxylic acid derivative having, in the ortho position of the carboxyl functional group, a leaving group, which is preferably an atom of fluorine or of chlorine or an alkoxy group, chiral or not, preferably a methoxy group, the carboxylic acid derivative not being substituted by an electro attractive group other than the leaving group if any; is reacted with a reactant MNu, where M is a metal and Nu is a nucleophile, chiral or not, the aromatic nucleophilic substitution reaction being carried out without a catalyst and without a step of protecting/deprotecting the acid functional group of the starting compound.

### FIELD OF THE INVENTION

This invention relates to the field of chemical synthesis, and in particular the invention proposes a new process enabling a nucleophilic aromatic substitution to be performed on aromatic carboxylic acid derivatives, in the absence of a catalyst in order, in particular, but not exclusively, to form symmetric or asymmetric biaryls.

### PRIOR ART

Nucleophilic aromatic substitution is a very commonly used chemical reaction, during which an atom attached to an aromatic cycle is substituted by a nucleophilic group. It makes it possible to prepare a wide variety of aromatic compounds, in particular pharmaceutical active principles, for example biphenyls.

Nucleophilic aromatic substitution, performed at an industrial level, is usually performed in the presence of catalysts involving precious metals, in particular palladium. However, for increased safety of patients, pharmaceutical regulations have been made considerably stricter in recent years in order to require the pharmaceutical industry to remove the maximum traces of these precious metals in the finished pharmaceutical active principles. As an example, the European Medicines Agency EMA (Agence Européenne d'Évaluation des Médicaments, EMEA) indicates for palladium a tolerated daily dose of 100 micrograms if the API is administered orally or 10 micrograms parenterally, i.e. less than 10 ppm and 1 ppm, respectively. In practice, when the synthetic pattern of the active principle requires the use of a precious metal at the end of synthesis and the metal content standards allowed for this active principle are exceeded, it is necessary to find removal processes, which costly both in time and money.

The trapping or removal of the residual metal catalysts is, for the pharmaceutical industry, a time-consuming and expensive step, capable of producing polluting residues, and there is a real need to overcome these constraints (see, for example, Königsberger et al, Organic Process Research & Development 2003, 7, 733-742, or Pink et al. Organic Process Research & Development 2008, 12, 589-595).

Another known disadvantage of nucleophilic substitution is the need to protect/deprotect the carboxyl function (CO₂H), necessary as a carbon anchoring point for subsequent chemical functionalization. It is indeed generally accepted that the CO₂H function reacts with organometallic compounds to lead to ketone derivatives (Jorgenson, M. J. Org. React. 1970, 18, 1. Ahn, T.; Cohen, T. Tetrahedron Lett. 1994, 35, 203). The protective group the most commonly used is the oxazoline function, and the reaction is known as the Meyers reaction (Meyers et al., Tetrahedron 2004, 60(20), 4459). According to this reaction, starting with a benzoic acid orthosubstituted by a fluorine atom or an alkoxy group, the carboxyl function is first protected (1→2, diagram 1). Aryloxazoline 2 thus obtained is capable of promoting the displacement of the ortho-alkoxy and fluoro groups by nucleophiles (“Nu”) (2→3, diagram 1). A step of deprotection of 3 must then be performed in order to release the CO₂H function and obtain the desired compound 4. The oxazoline may be chiral and the reaction with aryllithium or magnesium derivatives leads to optically active biaryls.

The Meyers reaction is of great industrial interest, in particular for obtaining these optically active biaryls, but requires these protection/deprotection steps. Moreover, the Meyers reaction does not make it possible to treat compounds 3 comprising a C6 substituent other than hydrogen: these compounds are totally inert to hydrolysis of the protected carboxyl group and do not lead to 4.

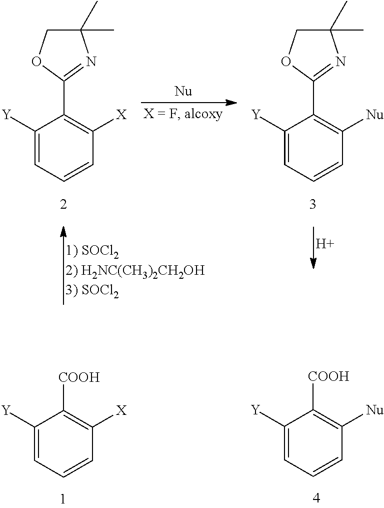

### Diagram 1

The invention proposes a new process that enables nucleophilic aromatic substitution, on an industrial scale and with a high yield, in an optimized number of steps. The invention has the industrial advantage of not requiring the use of metal catalysts, and therefore allows avoiding all of the current steps of purification/removal of precious metals, in particular palladium. It also has the advantage of not producing polluting residues. The invention has another advantage, which is that it does not require protection/deprotection step, for the starting compounds having a carboxyl function, for example but not exclusively benzoic acids, naphthoic acids and derivatives. Thus, the process according to the invention is a one-step process.

### DEFINITIONS

In the sense of this invention, the term “aryl” means a mono- or polycyclic system of 5 to 20, and preferably 6 to 12, carbon atoms having one or more aromatic rings (when there are two rings, it is called a biaryl) among which it is possible to cite the phenyl group, the biphenyl group, the 1-naphthyl group, the 2-naphthyl group, the tetrahydronaphthyl group, the indanyl group and the binaphthyl group. The term aryl also means any aromatic ring including at least one heteroatom selected from oxygen, nitrogen or sulfur atoms. The aryl group can be substituted by 1 to 3 substituents selected independently of one another from a hydroxyl group, a linear or branched alkyl group comprising 1, 2, 3 or 4, 5 or 6 carbon atoms, in particular methyl, ethyl, propyl, butyl, alkoxy group or halogen atom, in particular bromine, chlorine and iodine.

The term “catalyst” refers to any product involved in the reaction for increasing the speed of said reaction, but is regenerated or removed during or at the end of the reaction.

By “protecting the carboxyl function (CO₂H)”, we mean adding to said function a group destroying the reactivity of the carboxyl function with regard to nucleophiles; this group may be an oxazoline; numerous chemical groups other than the oxazoline function have been used to protect the CO₂H function: 2,6-di-tert-butyl-4-methoxyphenylic ester (Hattori, T.; Satoh, T.; Miyano, S. Synthesis 1996, 514. Koshiishi, E.; Hattori, T.; Ichihara, N.; Miyano, S. J. Chem. Soc., Perkin Trans. 1 2002, 377), amide (Kim, D.; Wang, L.; Hale, J. J.; Lynch, C. L.; Budhu, R. J.; MacCoss, M.; Mills, S. G.; Malkowitz, L.; Gould, S. L.; DeMartino, J. A.; Springer, M. S.; Hazuda, D.; Miller, M.; Kessler, J.; Hrin, R. C.; Carver, G.; Carella, A.; Henry, K.; Lineberger, J.; Schleif, W. A.; Emini, E. A. Bioorg. Med. Chem. Lett. 2005, 15(8), 2129), alkylamide (Guo, Z.; Schultz, A. G. Tetrahedron Lett. 2001, 42(9), 1603), dialkylamides (Hoarau, C.; Couture, A.; Deniau, E.; Grandclaudon, P. Synthesis 2000), 1-imidazolyles (Figge, A.; Altenbach, H. J.; Brauer, D. J.; Tielmann, P. Tetrahedron: Asymmetry 2002, 13(2), 137), 2-oxazolyles (Cram, D. J.; Bryant, J. A.; Doxsee, K. M. Chem. Lett. 1987, 19), 2-thiazolyles, etc.

By “leaving group” we mean a group that leads the two electrons of the sigma bond connecting it with the aromatic carbon atom during the substitution reaction with the nucleophile; according to the invention, the leaving group may be chiral or non-chiral; according to a preferred embodiment of the invention, the leaving group is chiral; according to the invention, the leaving group can be electron withdrawing or non-electron withdrawing.

By “alkyl”, we mean any saturated linear or branched hydrocarbon chain, with 1 to 12 carbon atoms, preferably 1 to 6 carbon atoms, and more preferably methyl, ethyl, propyl, isopropyl, n-butyl, sec-butyl, isobutyl and tert-butyl.

By “alkoxy”, we mean any O-alkyl or O-aryl group, chiral or not.

By “alkenyl”, we mean any linear or branched hydrocarbon chain having at least one double bond, of 2 to 12 carbon atoms, and preferably 2 to 6 carbon atoms.

By “alkynyl”, we mean any linear or branched hydrocarbon chain having at least one triple bond, of 2 to 12 carbon atoms, and preferably 2 to 6 carbon atoms.

By “amine”, we mean any compound derived from ammoniac NH₃ by substitution of one or more hydrogen atoms with an organic radical. According to the invention, a preferred amine is an aniline derivative.

By “functional group”, we mean a sub-molecular structure including an assembly of atoms conferring a specific reactivity to the molecule that bears it, for example an oxy, carbonyl, carboxy, sulfonyl group, etc.

By “nucleophile”, we mean an acyclic or cyclic compound, of which the characteristic is to include at least one atom with a free electron pair, charged or not. According to a preferred embodiment of the invention, we mean by “nucleophile” an acyclic or cyclic compound of which the characteristic is to include at least one atom with a charged free electron pair, preferably negatively charged.

By “nucleophile that may be chiral”, we mean a nucleophile with at least one asymmetric carbon.

By “electron withdrawing group” we mean a functional group having the ability to attract electrons, in particular if it is a substituent of an aromatic group, for example a group such as in particular of the NO₂ or SO₂R, in which R is alkyl, or CN or halogen. Amines and alkoxy groups are not electron withdrawing groups.

By “heterocycle”, we mean a 5- or 6-membered ring containing 1 to 2 heteroatoms chosen from O, S, N, optionally substituted with an alkyl.

By “aniline derivatine”, we mean a compound of general formula

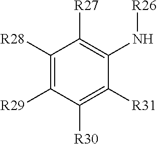

in which
R26 is a hydrogen atom, an alkyl group, an alkoxy group or an aryl;
R27, R28, R29, R30 and R31 are each independently a hydrogen atom, an halogen atom, an alkyl group, an aryl group, a heterocyclic group, a haloalkyl group, an alkoxy group, a nitro group, a cyano group or —(O)ₘ—(CH₂)ₙ—R32, or —[N(H)]ₘ—(CH₂)ₙ—R32, or two of these substituents bound to contiguous carbon atoms form an aryl ring, a heteroaryl ring, a heterocyclic group or a cycloalkyl group with 4 to 7 members,
or, when R27 is not in a ring with R28 and when neither R26 nor R27 are H, R26 and R27 may be member, with the nitrogen atom to which R26 is linked and with the contiguous carbon atom to this nitrogen atom, of a 5- or 6-membered ring, aromatic or dihydroaromatic, with carbon atoms and 1 or 2 nitrogen atoms,
with m equal to 0 or 1, n equal to 0, 1, 2, 3, or 4, and R32 is a hydrogen atom, a hydroxy group, —COOH or a disubstituted amine
According to the invention, alkylamines and dialkylamines are not aniline derivatives.

By “MNu”, we mean a reactant in which M is a metal and Nu is an independent nucleophile or a substituent of the aromatic ring of the benzoic acid derivative of general formula (II), said substituent being capable—or bearing a functional group capable—of reacting in the presence of a base and a metal to form MNu. When Nu is a substituent of the aromatic ring of (II), the nucleophilic aromatic substitution reaction occurs intramolecularly between the MNu function formed on the substituent and the leaving group in the ortho position of the carboxylic acid function.

### General Description

Thus, the invention relates to a process for preparing aromatic carboxylic acid derivatives, preferably benzoic acids, by nucleophilic aromatic substitution, in which the following are reacted:

an aromatic carboxylic acid derivative bearing a carboxyl function and a single one, or one of the salts thereof, preferably a lithium, sodium, potassium salt or a zinc salt, preferably a benzoic acid derivative or one of the salts thereof, said carboxylic acid derivative having, in the ortho position of the carboxyl function, a leaving group, which is preferably a fluorine or chlorine atom or a chiral or non-chiral alkoxy group, and in this last case, a methoxy group is preferred;

said carboxylic acid derivative being not substituted:
by another electron withdrawing group than the leaving group if any,
by a phenyl group, substituted in para position, especially by a benzyloxy in para position, when the leaving group is a fluorine or chlorine atom;

with a MNu reactant, in which M is a metal and Nu is a chiral or non-chiral nucleophile,

said nucleophilic aromatic substitution reaction being performed without catalyst and without a step of protection/deprotection of the acid function of the starting compound.

Preferably, the aromatic carboxylic acid derivative, starting compound of the reaction, is a benzoic acid derivative of general formula (II)

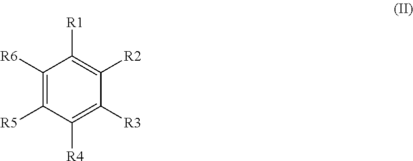

in which

R1 is CO₂H, and R2 is a fluorine or chlorine atom or an alkoxy group, chiral or not, preferably OCH₃,

or

R1 is a fluorine or chlorine atom or an alkoxy group, chiral or not, preferably OCH₃ and R2 is CO₂H

R3 is a hydrogen atom, an alkyl group, and alkoxy group, an aryl or an amine substituted or not by one or two alkyl groups, or R3 forms with R4 an aromatic ring or not, or a heterocycle, optionally substituted, in particular by a functional group; or is a substituent capable of reacting in presence of a base and a metal to form MNu;

R4 is a hydrogen atom, an alkyl group, an alkoxy group, preferably OCH₃, an aryl or an amine substituted or not by one or two alkyl groups, or R4 forms with R3 an aromatic ring or not, or a heterocycle, optionally substituted, in particular by a functional group, or R4 forms with R5 an aromatic ring or not, or a heterocycle, optionally substituted, in particular by a functional group; or is a substituent capable of reacting in presence of a base and a metal to form MNu;

R5 is a hydrogen atom, an alkyl group, an alkoxy group, an aryl or an amine substituted or not by one or two alkyl groups or R5 forms with R4 an aromatic ring or not, or a heterocycle, optionally substituted, in particular by a functional group, or R5 forms with R6 an aromatic ring or not, or a heterocycle, optionally substituted, in particular by a functional group; or is a substituent capable of reacting in presence of a base and a metal to form MNu;

R6 is a hydrogen atom, an alkyl group, an alkoxy group, an aryl or an amine substituted or not by one or two alkyl groups, or R6 forms with R5 and aromatic ring or not, or a heterocycle, optionally substituted, in particular by a functional group; or is a substituent capable of reacting in presence of a base and a metal to form MNu;

which reacts with

a compound (III) of general formula NuM in which Nu is a nucleophile, and M is a metal, preferably Li, Mg, Zn, Cu or an organomagnesium derivative MgX in which X is a halogen atom or an alkoxy group, chiral or not, preferably OCH₃,

said nucleophilic aromatic substitution reaction being performed without catalyst and without step of protection/deprotection of the acid function of the compound (II), in order to obtain a compound of general formula (I), which corresponds to the general formula (II) in which the R1 or R2 that is not CO₂H has been substituted by Nu.

### Procedure

Advantageously, the reaction is performed at between −78° C. and the solvent reflux. Preferably, the reaction is performed in a polar aprotic solvent, preferably anhydrous THF (tetrahydrofuran) or diethyl ether, benzene, toluene or a hydrocarbon such as pentane, hexane, heptane or octane.

Advantageously, NuM compound is preferably added dropwise, at a temperature comprised between −78° C. and solvent reflux.

Preferably, the solution is stirred, and then hydrolyzed with water. Advantageously, the hydrolysis is performed at low temperature. The pH is adjusted to 1 with an aqueous hydrochloric acid solution (2N) and the solution is extracted with an appropriate solvent, for example ethyl acetate. The organic phase is then dried and concentrated under vacuum. The raw product is recrystallized or chromatographied.

According to an embodiment of the invention, at least one equivalent of NuM is used for one equivalent of starting aromatic carboxylic acid derivative. Advantageously, in addition to this equivalent, one equivalent of NuM per leaving group of the starting molecule to be substituted is added.

According to another embodiment of the invention, at least one equivalent of a metal base, preferably butyllithium, sodium hydride, potassium hydride or lithium hydride is used for one equivalent of starting aromatic carboxylic acid derivative in order to form the metal salt corresponding to the acid function of the aromatic carboxylic acid derivative, and at least one equivalent of NuM is added per leaving group of the staring molecule to be substituted.

According to an embodiment, if the starting compound is a salt of aromatic carboxylic acid, at least one equivalent of NuM is used for one equivalent of salt of starting aromatic carboxylic acid derivative in order to form the metal salt corresponding to the acid function and at least one equivalent of NuM is added per leaving group of the starting molecule to be substituted.

According to another embodiment, if the starting compound is a salt of aromatic carboxylic acid, at least one equivalent of a metal base, preferably butyllithium, sodium hydride, potassium hydride or lithium hydride is used for an equivalent of salt of starting aromatic carboxylic acid derivative in order to form the metal salt corresponding to the acid function, and at least one equivalent of NuM is added per leaving group of the staring molecule to be substituted.

The yields expected for the reaction process according to the invention are between 40 and 100%, preferably 45 to 90%, and more preferably 60 to 90%.

### Specific Cases

According to a first preferred embodiment, R1 is CO₂H, R2 is an alkoxy, preferably OCH₃, and R3 to R6 are as defined above.

According to a second preferred embodiment, if R2 is CO₂H, R1 is an alkoxy, preferably OCH₃ and R3 to R6 are as defined above.

According to another embodiment, a hydrogen atom is in para position of the acid function. According to a first embodiment, if R1 is CO₂H, R4 is a hydrogen atom and R2, R3, R5 and R6 are as defined above. According to a second embodiment, if R2 is CO₂H, R5 is a hydrogen atom and R1, R3, R4 and R6 are as defined above.

According to a specific embodiment of the process according to the invention, the compound of general formula (II) is such that R1 is CO₂H, R2 is a halogen atom, preferably fluorine or an alkoxy group, chiral or not, preferably methoxy, and R3 to R6 are as defined above and are preferably each a hydrogen atom.

According to another specific embodiment of the process according to the invention, compound of general formula (II) is such that R1 is CO₂H, R2 is a halogen atom, preferably fluorine, or an alkoxy group, chiral or not, preferably methoxy, R3 and R4, or R4 and R5, or R5 and R6 form together a ring, optionally substituted, such that the starting aromatic carboxylic acid derivative is a naphthalene derivative of general formulae (IIa, IIb or IIc) below, in which R7, R8, R9 and R10 are each independently a hydrogen atom, an alkyl group, an alkoxy group, an aryl or an amine substituted or not by one or two alkyl groups; and substituents R3, R4, R5 and R6 not member of in the ring are as defined above.

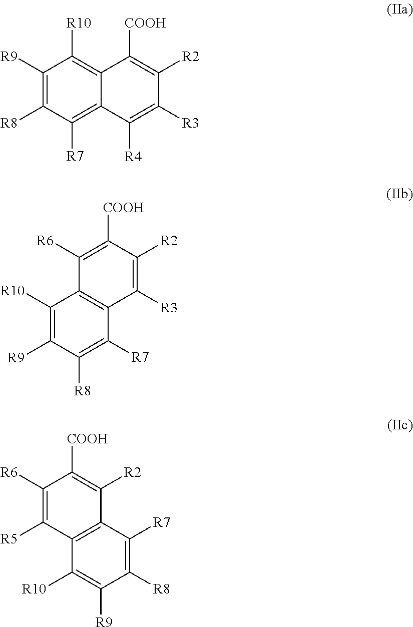

According to a preferred embodiment, when the leaving group is fluorine, MNu is not sBuLi or tBuLi or PhLi.

According to another preferred embodiment, when the leaving group is a methoxy, MNu is not sBuLi.

### Presence of an Asymmetric Carbon

According to a preferred embodiment, an asymmetric carbon is present on said aromatic carboxylic acid derivative, starting compound of the reaction, preferably on said benzoic acid derivative of general formula (II) and/or on the nucleophile, and the compound of general formula (I) obtained is asymmetric. Very advantageously, the aromatic acid derivative, preferably on said benzoic acid derivative of general formula (II), has at least one chiral leaving group.

According to another specific embodiment, an asymmetric carbon is present in the leaving group of the aromatic carboxylic acid derivative and/or on the nucleophile, and the compound of general formula (I) obtained is asymmetric.

### Use of a Chiral Ligand

In a specific embodiment, the reaction medium has a chiral ligand added to it; this ligand is intended to induce chirality to the product (I) of the reaction of the invention.

According to the invention, said chiral ligand may be chosen from the chiral diamines, the chiral diethers, the chiral aminoethers, the multi-point binding chiral aminoethers and the bisoxazoline ligands. Examples of chiral ligands that may be used are depicted in table 1.

TABLE 1

| | |
|---|---|
| Example of chiral diamine | 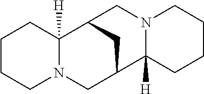 |
| Example of chiral diether | 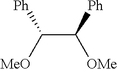 |
| Example of chiral aminoether | 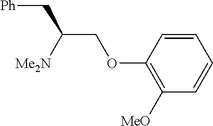 |
| Example of multi-point binding chiral aminoether | 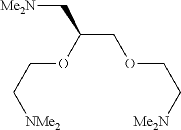 |
| Example of bisoxazoline ligand | 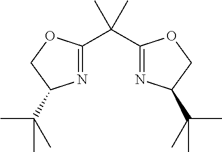 |

Case in which the Leaving Group is a Fluorine or a Chlorine Atom

According to a first embodiment, when a fluorine or a chlorine atom is in the ortho position of the acid function, Nu is not a substituted or non-substituted amine, especially Nu is not an aniline derivative, more especially Nu is not 4-[2-(3,4-dichlorophenyl)ethyl]aniline.

According to a second embodiment, when a fluorine atom is in ortho position of the acid function, Nu is not a substituted or non-substituted amine

According to an embodiment of the invention, compound (II) is such that the leaving group (R1 or R2) is a fluorine or chlorine atom, and the nucleophile of the compound of general formula NuM is an aniline derivative. In this embodiment, according to a first aspect, NuM compound is obtained according to the synthesis modes described below, given that NuM is not the product of a reaction between the nucleophile and a metal base selected from lithium hydride, sodium hydride, potassium hydride, calcium hydride, lithium diisopropylamide, lithium amide, sodium amide, potassium amide, sodium methoxide, sodium ethoxide, potassium tert-butoxide, magnesium ethoxide and LiHMDS. In this embodiment, according to a second aspect, NuM compound is obtained by a reaction of nucleophile and butyllithium.

### Obtaining the NuM Compound (III)

According to a first embodiment, the compound NuM may be obtained by direct synthesis (Carey & Sundberg, Advanced Organic Chemistry, Part A Chapter 7, “Carbanions and Other Nucleophilic Carbon Species”, pp. 405-448).

According to a second embodiment, compound NuM may be obtained from lithium salts and anion radicals (T. Cohen et al. JACS 1980, 102, 1201; JACS 1984, 106, 3245; Acc. Chem. Res, 1989, 22, 52).

According to a third embodiment, compound NuM may be obtained by metal-halogen exchange (Parham, W. E.; Bradcher, C. K. Acc. Chem. Res. 1982, 15, 300-305).

According to a fourth embodiment, the compound NuM can be obtained by directed metallization (V. Snieckus, Chem. Rev, 1990, 90, 879; JOC 1989, 54, 4372).

According to a preferred embodiment of the invention, the compound NuM is obtained by reaction of the nucleophile and a base, in particular a metal or an organometallic base. According to a first embodiment, the base is not LiHMDS or a mixture of lithium hydride and diethoxyethane. According to a second embodiment, the metal base is not chosen from the group consisting of lithium hydride, sodium hydride, potassium hydride, calcium hydride, lithium diisopropylamide, lithium amide, sodium amide, potassium amide, sodium methoxide, sodium ethoxide, potassium tert-butoxide, magnesium ethoxide, and LiHMDS. According to a third embodiment, the base is butyllithium, and in this embodiment, advantageously, NuM compound is obtained by a reaction of the nucleophile and n-BuLi, tert-BuLi or sec-BuLi. According to a fourth embodiment, the base is chiral and induces chirality to NuM.

Preferably, Nu is a nucleophile chosen from those described in tables 2, 3 and 4.

Tables 2, 3 and 4 below show a plurality of preferred NuM reactants.

TABLE 2

| Nu | M |
|---|---|
| Alkyl, preferably CH₃ or C₂H₅ | Li, Mg, Cu, Zn, or MgX in which X is a |
| | halogen or an alkoxy |
| Alkenyl, optionally substituted | Li, Mg, Cu, Zn, or MgX in which X is a |
| | halogen or an alkoxy |
| Alkynyl optionally substituted | Li, Mg, Cu, Zn, or MgX in which X is a |
| | halogen or an alkoxy |
| Aryl optionally substituted | Li, Mg, Cu, Zn, or MgX in which X is a |
| | halogen or an alkoxy |
| s-Bu | Li, Mg, Cu, Zn, or MgX in which X is a |
| | halogen or an alkoxy |
| t-Bu | Li, Mg, Cu, Zn, or MgX in which X is a |
| | halogen or an alkoxy |
| n-Bu | Li, Mg, Cu, Zn, or MgX in which X is a |
| | halogen or an alkoxy |
| 4-MeOC₆H₄ | Li, Mg, Cu, Zn, or MgX in which X is a |
| | halogen or an alkoxy |
| 2-MeOC₆H₄ | Li, Mg, Cu, Zn, or MgX in which X is a |
| | halogen or an alkoxy |
| 2,5-diMeC₆H₄ | Li, Mg, Cu, Zn, or MgX in which X is a |
| | halogen or an alkoxy |
| 4-Me₂NC₆H₄ | Li, Mg, Cu, Zn, or MgX in which X is a |
| | halogen or an alkoxy |
| 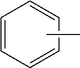 | Li, Mg, Cu, Zn, or MgX in which X is a halogen or an alkoxy |
| 2-MeC₆H₄ | Li, Mg, Cu, Zn, or MgX in which X is a |
| | halogen or an alkoxy |
| 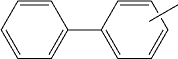 | Li, Mg, Cu, Zn, or MgX in which X is a halogen or an alkoxy |
| 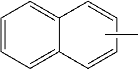 | Li, Mg, Cu, Zn, or MgX in which X is a halogen or an alkoxy |
| 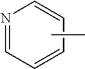 | Li, Mg, Cu, Zn, or MgX in which X is a halogen or an alkoxy |
| 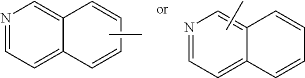 | Li, Mg, Cu, Zn, or MgX in which X is a halogen or an alkoxy |
| 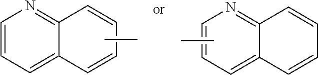 | Li, Mg, Cu, Zn, or MgX in which X is a halogen or an alkoxy |
| 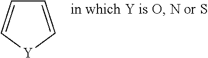 | Li, Mg, Cu, Zn, or MgX in which X is a halogen or an alkoxy |
| 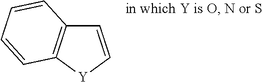 | Li, Mg, Cu, Zn, or MgX in which X is a halogen or an alkoxy |
| P(Aryl)₂, | Li, Mg, Cu, Zn, or MgX in which X is a |
| | halogen or an alkoxy |
| PArylAlkyl | Li, Mg, Cu, Zn, or MgX in which X is a |
| | halogen or an alkoxy |
| O(C₁₋₆alkyl) | Li, Mg, Cu, Zn, or MgX in which X is a |
| | halogen or an alkoxy |
| S(C₁₋₆alkyl) | Li, Mg, Cu, Zn, or MgX in which X is a |
| | halogen or an alkoxy |
| 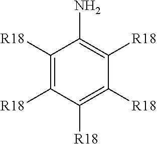 in which R18 is a hydrogen atom, an alkyl group, an alkoxy group, an aryl or an amine substituted or not by one or two C₁₋₁₂alkyl groups | Li, Mg, Cu, Zn, or MgX in which X is a halogen or an alkoxy |

TABLE 3

| Nu | M |
|---|---|
| N(C₁₋₆alkyl)₂ | Li, Mg, Cu, Zn, or MgX in which X is a halogen |
| | or an alkoxy |
| NH(C₁₋₆alkyl), in | Li, Mg, Cu, Zn, or MgX in which X is a halogen |
| particular NH(tBu) | or an alkoxy |
| NEt₂ | Li, Mg, Cu, Zn, or MgX in which X is a halogen |
| | or an alkoxy |
| 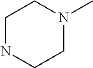 | Li, Mg, Cu, Zn, or MgX in which X is a halogen or an alkoxy |
| N(iPr)₂ | Li, Mg, Cu, Zn, or MgX in which X is a halogen |
| | or an alkoxy |
| 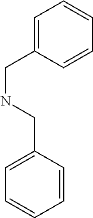 | Li, Mg, Cu, Zn, or MgX in which X is a halogen or an alkoxy |
| 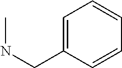 | Li, Mg, Cu, Zn, or MgX in which X is a halogen or an alkoxy |
| 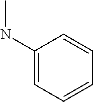 | Li, Mg, Cu, Zn, or MgX in which X is a halogen or an alkoxy |
| N(CH₂CH₂)₂NMe | Li, Mg, Cu, Zn, or MgX in which X is a halogen |
| | or an alkoxy y |
| NMeBn | Li, Mg, Cu, Zn, or MgX in which X is a halogen |
| | or an alkoxy |
| NBn₂ | Li, Mg, Cu, Zn, or MgX in which X is a halogen |
| | or an alkoxy |
| NMePh | Li, Mg, Cu, Zn, or MgX in which X is a halogen |
| | or an alkoxy |
| NHt-Bu | Li, Mg, Cu, Zn, or MgX in which X is a halogen |
| | or an alkoxy |
| NPh₂ | Li, Mg, Cu, Zn, or MgX in which X is a halogen |
| | or an alkoxy |

According to a first preferred embodiment of the invention, in tables 2 and 3, M is Li or Mg.

According to a preferred embodiment, M is Li, Mg, Cu, Zn, or MgX in which X is a halogen or an alkoxy and Nu is N(C₁₋₆alkyl)₂, NH(C₁₋₆alkyl), NEt₂, N(CH₂CH₂)₂NMe, NMeBn, NBn₂, NMePh, NHt-Bu or NPh₂.

Advantageously, in tables 2 and 3, when M is MgX with X being halogen, the halogen is chosen from F, Br, Cl. Advantageously, when M is MgX with X being alkoxy, the alkoxy is OCH₃ or OC₂H₅. According to a preferred embodiment of the invention, M is MgBr or MgOCH₃.

The preferred chiral NuM compounds according to the invention are depicted as examples in table 4 below.

TABLE 4

| Nu | M |
|---|---|
| 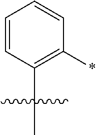 | Li, Mg |
| 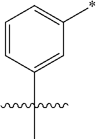 | Li, Mg |
| 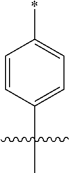 | Li, Mg |
| 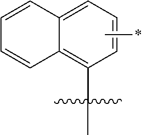 | Li, Mg, Cu, Zn |
| 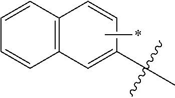 | Li, Mg, Cu, Zn |
| 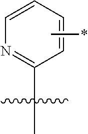 | Li, Mg, Cu, Zn |
| 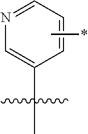 | Li, Mg, Cu, Zn |
| 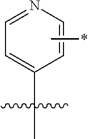 | Li, Mg, Cu, Zn |
| 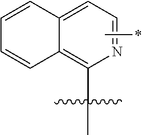 | Li, Mg, Cu, Zn |
| 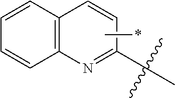 | Li, Mg, Cu, Zn |
| 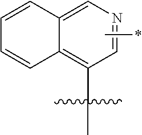 | Li, Mg, Cu, Zn |
| 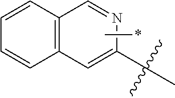 | Li, Mg, Cu, Zn |
| 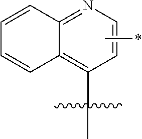 | Li, Mg, Cu, Zn |
| 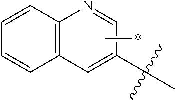 | Li, Mg, Cu, Zn |
| 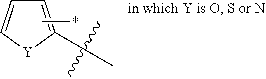 | Li, Mg |
| 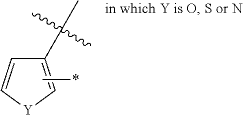 | Li, Mg |
| 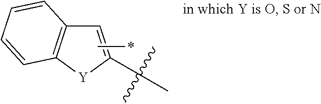 | Li, Mg |
| 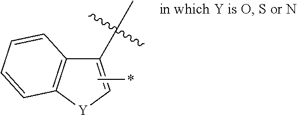 | Li, Mg |
| NR¹¹R¹²* in which R¹¹ and R¹² are each | Li, Mg |
| independently a hydrogen atom, an alkyl | |
| group, an alkoxy group, an aryl, or an | |
| amine substituted or not by one or two | |
| C₁₋₁₂alkyl groups. | |
| SiR¹³R¹⁴R¹⁵* in which R13, R14 and R15 | Li, Mg |
| are each independently a hydrogen atom, | |
| an alkyl group, an alkoxy group, an aryl, | |
| or an amine substituted or not by one or | |
| two C₁₋₁₂alkyl groups. | |
| OR¹⁶* in which R¹⁶ is a hydrogen atom, an | Li, Mg |
| alkyl group, an alkoxy group, an aryl, or | |
| an amine substituted or not by one or two | |
| C₁₋₁₂alkyl groups. | |
| SR¹⁷* in which R¹⁷ is a hydrogen atom, an | Li, Mg |
| alkyl group, an alkoxy group, an aryl, or | |
| an amine substituted or not by one or two | |
| C₁₋₁₂alkyl groups | |

*chiral element

According to a specific embodiment of the invention, each non-substituted position of an aromatic ring of one of tables 2 to 4 may be substituted by a hydrogen atom, an alkyl group, an alkoxy group, an aryl, or an amine substituted or not by one or two C1-12alkyl groups.

### Use of (I) to Obtain a Benzo[c]Phenantridine

According to a preferred embodiment, the obtained compound of formula (I) allows then obtaining a benzo[c]phenantridine. Examples of benzo[c]phenantridine susceptible of being obtained by a reaction implementing in particular a nucleophilic aromatic substitution are provided in table 5 below:

TABLE 5

| | |
|---|---|
| benzo[c] phenanthridine | 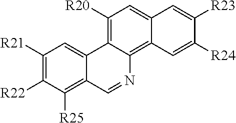 |
| benzo[c][1,7] phenanthroline | 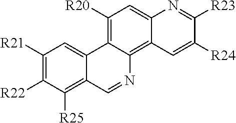 |
| benzo[c][1,8] phenanthroline | 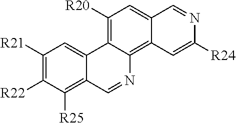 |
| benzo[c][1,9] phenanthroline | 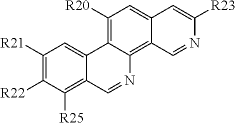 |
| benzo[c][1,10] phenanthroline, | 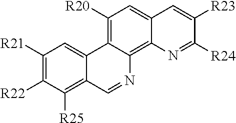 |
| pyridazino[4,5-c] phenanthridine | 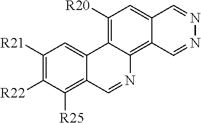 |

In all compounds of table 5 above, substituents R20, R21, R22, R23, R24 and R25 are each independently a hydrogen atom, an alkyl group, an alkoxy group, an aryl, or an amine substituted or not by one or two C₁₋₁₂alkyl groups.

Advantageously, compound of formula (I) obtained allows then obtaining fagaronine or ethoxidine, of which the formulae are depicted in table 6.

TABLE 6

| | |
|---|---|
| fagaronine | 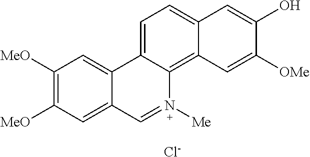 |
| Ethoxidine | 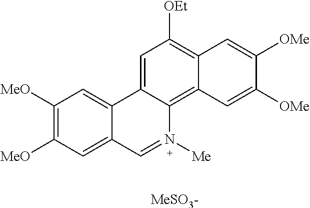 |

According to an embodiment of the invention, the reaction implementing in particular a nucleophilic aromatic substitution and allowing obtaining these compounds has the following route:

NuM+(II)→(I)→benzo[c]phenantridine

According to a first embodiment of the invention, NuM compounds, (II) and (I) are as defined in table 7 below:

TABLE 7

| NuM | II | I | Benzo[c]phenantridine |
|---|---|---|---|
| 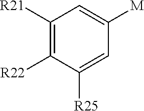 | 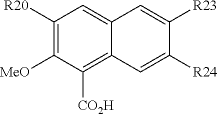 | 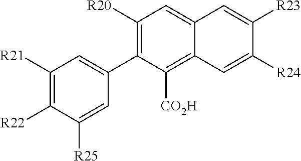 | benzo[c]phenanthridine |
| 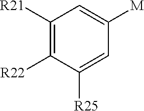 | 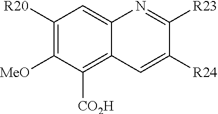 | 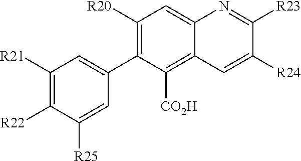 | benzo[c][1,7]phenanthroline |
| 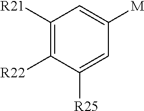 | 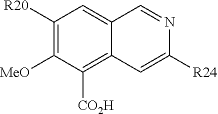 | 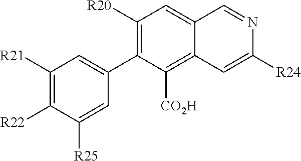 | benzo[c][1,8]phenanthroline |
| 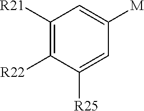 | 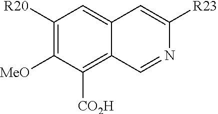 | 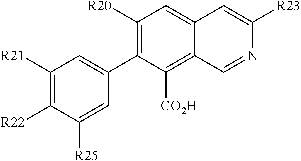 | benzo[c][1,9]phenanthroline |
| 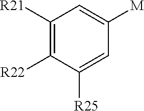 | 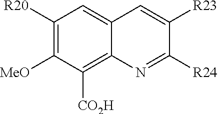 | 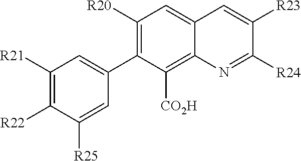 | benzo[c][1,10]phenanthroline |
| 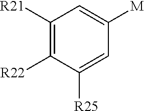 | 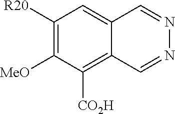 | 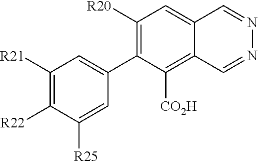 | pyridazino[4,5-c]phenanthridine |

In each compound of table 7, M is Li or Mg, and R20, R21, R22, R23, R24 and R25 are each independently a hydrogen atom, an alkyl group, an alkoxy group, an aryl, or an amine substituted or not by one or two C₁₋₁₂alkyl groups.

Thus, according to a preferred embodiment, the process leads to a product of formula (I) which is benzo[c]phenanthridine, benzo[c][1,7]phenanthroline, benzo[c][1,8]phenanthroline, benzo[c][1,9]phenanthroline, benzo[c][1,10]phenanthroline, pyridazino[4,5-c]phenanthridine.

According to a second embodiment of the invention, the NuM compounds (II) and (I) are as defined in table 8 below:

TABLE 8

| NuM | II | I | Benzo[c]phenantridine |
|---|---|---|---|
| 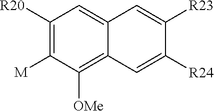 | 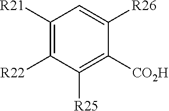 | 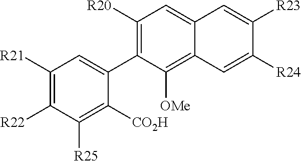 | benzo[c]phenanthridine |
| 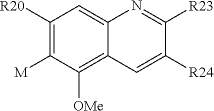 | 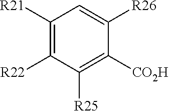 | 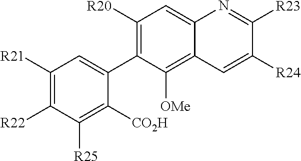 | benzo[c][1,7] phenanthroline |
| 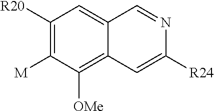 | 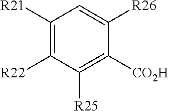 | 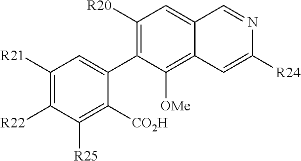 | benzo[c][1,8] phenanthroline |
| 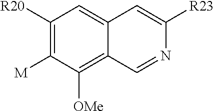 | 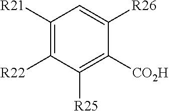 | 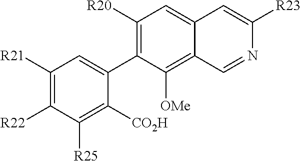 | benzo[c][1,9] phenanthroline |
| 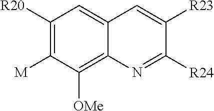 | 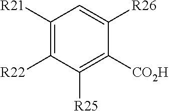 | 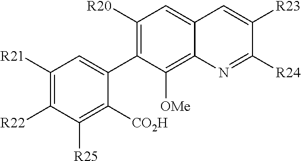 | benzo[c][1,10] phenanthroline |
| 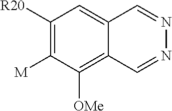 | 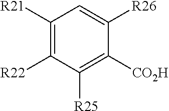 | 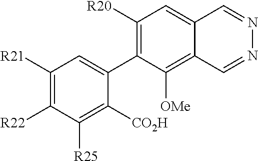 | pyridazino[4,5-c] phenanthridine |

In each compound of table 8, M is Li or Mg, and R20, R21, R22, R23, R24 and R25 are each independently a hydrogen atom, an alkyl group, an alkoxy group, an aryl, or an amine substituted or not by one or two C₁₋₁₂alkyl groups.

According to a preferred embodiment, the product of formula (I) is apogossypol, gossypol or a derivative of thereof, obtained by reaction of the following compound of formula (IId) with the following NuM:

| (IId) | NuM |
|---|---|
| 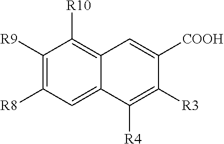 in which R4, R8, R9 are each independently an alkoxy group and R3 is an alkoxy or fluorine group with an asymmetric carbon | 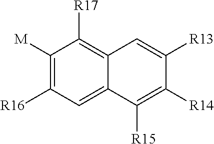 in which R13, R14, and R17 are each independently an alkoxy group and R15 and R16 are each independently an alkyl group |

The invention may be better understood in view of the following examples, which illustrate the process according to the invention in a non-limiting manner

### EXAMPLES

All of the reactions are performed under inert atmosphere with anhydrous solvents (Gordon, J. A.; Ford, R. A. The Chemist's Companion, Wiley J. and Sons, New York, 1972). The THF is distilled by means of an anhydrous THF GTS100 station (Glass Technology). Alkyllithium derivatives are periodically titrated with N-benzylbenzamide (Burchat, A. F.; Chong, J. M.; Nielsen, N. J. Organomet. Chem. 1997, 542, 281).

S-butyllithium (1.4 M in solution in cyclohexane), n-butyllithium (1.6 M in solution in hexane), t-butyllithium (1.7 M in solution in pentane) and phenyllithium (1.8 M in solution in dibutylether) are sold by Acros Chemicals and Aldrich Chemical Company.

Ethylmagnesium bromide (3 M in solution in diethylether) and vinylmagnesium bromide (1M in solution in THF) are sold by Acros Chemicals and Aldrich Chemical Company.

The amines are distilled over CaH₂ and stored under argon atmosphere.

The nuclear magnetic resonance spectra of the proton ¹H (400 MHz or 200 MHz) and of the carbon ¹³C (50 MHz or 100.6 MHz) were performed on a Bruker AC 400 or DPX 200 apparatus. The chemical shifts δ are given in parts per million (ppm).

Tetramethylsilane (TMS) is used as an internal reference when CDCl₃ is used as a solvent. In the case of acetone-d₆ and DMSO d₆, the chemical shifts are given with respect to the signal of the solvent. Coupling constants are given in Hertz (Hz). The following abbreviations are used to describe the NMR spectra: s (singlet), d (doublet), dd (double doublet), t (triplet), q (quadruplet), m (multiplet), sept (septuplet).

The mass spectra were recorded in chemical impact mode or in field ionization mode on a high-resolution spectrometer (GCT First High-Resolution Micromass). The precision obtained for the precise mass measurements is four digits.

Elemental analyses were performed by the microanalysis center of ICSN of -Gif sur Yvette. The infrared spectra were recorded on a Nicolet® Avatar® 370 DTGS spectrometer. The melting points were measured on a Büchi Melting Point B-540 apparatus.

1. SNArAB Reaction with Amides

### General Procedure for Preparation of Lithium Amide

To an amine solution (primary or secondary, n mmol) in anhydrous THF (m mL) is added dropwise n-BuLi (1.6 M in hexane, n mmol), at −30° C. for the secondary amine and at 0° C. for the primary amine. For the primary amines, the solution is stirred at 0° C. for 30 min then at room temperature for 1 h before use. In the case of the secondary amines, the solution is stirred at 0° C. for 30 min before use.

### Preparation of Anthranilic Acids

### 2-(diethylamino)benzoic acid (3)

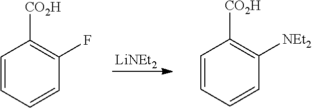

2-fluorobenzoic acid (420 mg, 3 mmol) 1 or 2-methoxybenzoic acid 2 (456 mg, 3 mmol) in solution in anhydrous THF (5 mL) is added dropwise at −50° C. to a lithium diethylamidide solution (6.6 mmol, prepared according to the general procedure in 12 mL of THF). The solution is stirred at −50° C. for 14 h for acid 1 while for acid 2, the solution is allowed to slowly warm up to 0° C. The reaction mixture is then hydrolyzed at 0° C. with distilled water (30 mL). The pH of the aqueous phase is adjusted to 7 by adding an aqueous HCl solution (2M) and the solution is extracted by dichloromethane (3*50 mL). The combined organic phases are dried over MgSO₄, filtered and concentrated under reduced pressure. After recrystallization (benzene/n-hexane 9/1), 2-(diethylamino)benzoic acid 3 is as a white solid (425 mg, 73% from 1; 541 mg, 93% from 2). Mp=122.4-123.0° C. (Haslam, J. L.; Eyring, E. M. J. Phys. Chem. 1967, 71(13), 4470.120-121° C.). ¹H NMR (200 MHz, CDCl₃) δ: 8.34 (dd, J=1.5 Hz, J=8 Hz, 1H, H₆), 7.62 (dt, J=1.3 Hz, J=8 Hz, 1H, H₄), 7.47-7.35 (m, 2H, H₅, H₃), 3.20 (m, 4H, 2*CH₂), 1.06 (t, J=7 Hz, 6H, 2*CH₃). ¹³C NMR (50 MHz, CDCl₃) δ: 167.9; 146.9; 133.8; 131.5; 128.0; 127.8; 122.4; 51.1; 11.6. IR (ATR, cm⁻¹): 2972, 1653, 1205. HRMS m/z calculated for C₁₁H₁₆NO₂ ([M+H]⁺): 194.1181. Found: 194.1176. Microanalysis calc. for C₁₁H₁₆NO₂: C, 68.37; H, 7.82; N, 7.25. Found: C, 68.39; H, 7.77; N, 7.17.

### 2-(4-Methylpiperazin-1-yl)benzoic acid (4)

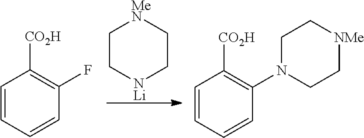

2-fluorobenzoic acid (420 mg, 3 mmol) 1 or 2-methoxybenzoic acid 2 (456 mg, 3 mmol) in solution in anhydrous THF (5 mL), respectively at −50° C. and 0° C. is added dropwise to a lithium (4-methylpiperazin-1-yl)amide solution (6.6 mmol, prepared according to the general procedure in 12 mL). The reaction mixture is stirred for 14 h at −50° C. for 1 and at 0° C. for 2 before being hydrolyzed at 0° C. by distilled water (30 mL). The pH of the aqueous phase is adjusted to 1 by the addition of an HCl solution (2M). The aqueous phase is extracted by ethyl acetate (3*50 mL). The aqueous phase is adjusted to pH=6 with an aqueous NaOH solution (2M) and concentrated under reduced pressure. The residue is dissolved in dichloromethane (300 mL) and stirred overnight. After filtration, the solution is dried over MgSO₄ and concentrated under reduced pressure. After recrystallization, acid 4 is isolated as a white solid (583 mg, 88% from 1 and 464 mg, 70% from 2). Mp=211-215° C. ¹H NMR (200 MHz, CDCl₃) δ: 8.30 (dd, J=1.96 Hz J=7.7 Hz, 1H, H₆), 7.60 (m, 1H, H₄), 7.41 (m, 2H, H₃, H₅), 3.10 (t, J=4.8 Hz, 4H, 2*CH₂), 2.70 (m, 4H, 2*CH₂), 2.40 (s, 3H, CH₃). ¹³C NMR (50 MHz, CDCl₃) δ: 166.9; 150.29; 133.9; 132.3; 127.6; 125.1; 122.4; 54.9; 53.4; 45.8. IR (ATR, cm⁻¹): 3063, 2975, 1657, 1231. HRMS m/z calculated for C₁₂H₁₇N₂O₂ ([M+H]⁺): 221.1290. Found: 221.1296. Microanalysis calc. For C₁₂H₁₇N₂O₂: C, 65.43; H, 7.32; N, 12.72. Found: C, 65.14; H, 7.48; N, 12.71.

### 2-(N-benzyl-N-methylamino)benzoic acid (5)

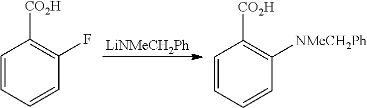

2-fluorobenzoic acid (420 mg, 3 mmol) 1 or 2-methoxybenzoic acid 2 (456 mg, 3 mmol) in solution in anhydrous THF (respectively 5 mL and 3.4) is added dropwise at −50° C. to a lithium N-benzyl-N-methylamide solution (2 equiv., prepared according to the general procedure at a concentration of 0.5 M). The solution is stirred at −50° C. for 14 h for acid 1 while for acid 2, the solution is allowed to slowly warm up to 0° C. The reaction mixture is then hydrolyzed at 0° C. with distilled water (respectively 30 mL and 20 mL). The pH of the aqueous phase is adjusted to 1 by the addition of an HCl solution (2M), and the aqueous phase is extracted with dichloromethane (3*50 mL). The combined organic phases are dried over MgSO₄, filtered and concentrated under reduced pressure. After recrystallization (MeOH/H₂O 6/4), acid 5 is isolated as a white solid (617 mg, 85% from 1; 316 mg, 65% from 2). Mp=86-88° C. ¹H NMR (200 MHz, CDCl₃) δ: 8.29 (dd, J=1.7 Hz, J=7.9 Hz, 1H, H₆), 7.64-7.33 (m, 8H, H arom), 4.11 (s, 2H, CH₂), 2.72 (s, 3H, CH₃). ¹³C NMR (50 MHz, CDCl₃) δ: 167.1; 150.9; 134.1; 133.8; 132.1; 129.8; 128.7; 128.6; 127.6; 125.5; 122.8; 62.6; 42.6. IR (ATR, cm⁻¹): 3059, 1690, 1220. HRMS m/z calculated for C₁₅H₁₅NO₂ ([M+H]⁺): 242.1181. Found: 242.1175. Microanalysis calc. for C₁₅H₁₅NO₂: C, 74.67; H, 6.27; N, 5.81. Found: C, 74.78; H, 6.23; N, 5.86.

### 2-(dibenzylamino)benzoic acid (6)

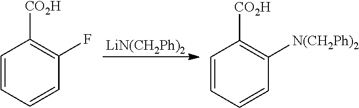

2-fluorobenzoic acid 1 (420 mg, 3 mmol) in solution in anhydrous THF (10 mL) is added dropwise at −50° C. to a lithium dibenzylamide solution (6.6 mmol, prepared according to the general procedure in 12 mL of THF). The solution is stirred at −50° C. for 14 h. The reaction mixture is then hydrolyzed at 0° C. with distilled water (30 mL). The pH of the aqueous phase is adjusted to 1 by the addition of an HCl solution (2M) in order to precipitate the excess dibenzylamine. The solution is filtered and extracted with dichloromethane (3*50 mL). The combined organic phases are dried on MgSO₄, filtered and concentrated under reduced pressure. After recrystallization (Et₂O), acid 6 is isolated as a white solid (763 mg, 80%). Mp=102-104° C. ¹H NMR (200 MHz, CDCl₃) δ: 8.15 (dd, J=1.6 Hz, J=7.8 Hz, 1H, H₆), 7.62-7.54 (m, 1H, H₄), 7.49-7.44 (m, 1H, H₅), 7.37-7.16 (m, 11H) 4.16 (s, 4H). ¹³C NMR (50 MHz, CDCl₃) δ: 166.8; 148.6; 134.0; 133.3; 132.0; 130.5; 130.0; 129.2; 129.0; 128.7; 128.4; 127.5; 126.7; 124.1; 60.1. IR (ATR, cm⁻¹): 3024, 1681, 1292. HRMS (EI) m/z calculated for C₂₁H₂₀NO₂ ([M+H]⁺): 318.1494. Found: 318.1471. Microanalysis calc. For C₂H₂₀NO₂: C, 79.47; H, 6.03; N, 4.41. Found: C, 79.55; H, 6.07; N, 4.45.

### 2-(N-methyl-N-phenylamino)benzoic acid (7)

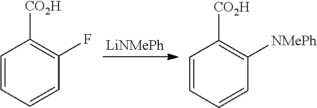

2-fluorobenzoic acid (280 mg, 2 mmol) in solution in anhydrous THF (3.5 mL) is added dropwise at room temperature to a lithium N-methyl-N-phenylamide solution (4.2 mmol, prepared according to the general procedure in 8 mL of THF). The solution is then stirred at 60° C. for 3.5 h and the reaction mixture is hydrolyzed at room temperature with distilled water (20 mL). The pH of the aqueous phase is adjusted to lupon addition of an HCl solution (2M) and the aqueous phase is extracted by dichloromethane (3*50 mL). The combined organic phases are dried over MgSO₄, filtered and concentrated under reduced pressure. After recrystallization (Et₂O/petroleum ether 7/3), acid 7 is isolated as a green solid (409 mg, 60%). Mp: 103-107° C. (Coombs, R. V. J. Org. Chem. 1977, 42(10), 1812-1813 104-104.5° C.). ¹H NMR (200 MHz, CDCl₃) δ: 8.40 (dd, J=0.43 Hz, J=7.8 Hz, 1H, H₆), 7.62-7.40 (m, 2H), 7.39-7.20 (m, 2H), 7.18-7.05 (m, 2H), 7.00-6.90 (m, 2H), 3.23 (s, 3H). IR (ATR): 2815, 1681, 1297 cm⁻¹.

### 2-(diphenyl)amino)benzoic acid (8)

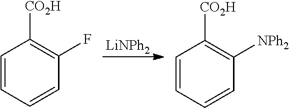

2-fluorobenzoic acid (280 mg, 2 mmol) in solution in anhydrous THF (3.5 mL) is added dropwise at room temperature to a lithium diphenylamide solution (4.4 mmol, prepared according to the general procedure in 8 mL of THF). The solution is then stirred at 60° C. for 72 h and the reaction mixture is hydrolyzed at room temperature with distilled water (30 mL). The pH of the aqueous phase is adjusted to 5 upon addition of an HCl solution (2M) and the aqueous phase is extracted by ethyl acetate (3*50 mL). The combined organic phases are dried over MgSO₄ and concentrated under reduced pressure. Acid 8 is isolated as a green solid (416 mg, 70% conversion). ¹H NMR (200 MHz, CDCl₃) δ: 7.95 (dd, J=1.7 Hz, J=7.8 Hz, 1H, H₆), 7.50 (td, J=1.8 Hz, J=7.7 Hz, 1H, H₄), 7.30-7.10 (m, 6H, H arom) 7.00-6.85 (m, 6H, H arom).

### 2-(diisopropylamino)benzoic acid (9)

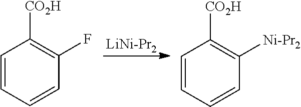

2-fluorobenzoic acid 1 (420 mg, 3 mmol) in solution in anhydrous THF (5 mL) is added dropwise to a lithium diisopropylamide solution (6.6 mmol, prepared according to the general procedure in 12 mL of THF). The reaction mixture is stirred for 14 h at −50° C. for 1 and at 0° C. for 2 before being hydrolyzed at 0° C. by distilled water (30 mL). The pH of the aqueous phase is adjusted to 8/9 upon addition of an HCl solution (2M) and the solution is extracted with dichloromethane (3*50 mL). The combined organic phases are dried over MgSO₄, filtered and concentrated under reduced pressure. After recrystallization (Et₂O/cyclohexane 55/45), the acid (9) is isolated as a white solid (186 mg, 28%). Mp=90.5-91.5° C. ¹H NMR (200 MHz, CDCl₃) δ: 8.37 (dd, J=1.9 Hz, J=7.6 Hz, 1H, H₆), 7.60-7.40 (m, 2H, H₅ and H₄), 7.29 (dd, J=1.4 Hz, J=7.6 Hz, 1H, H₃), 3.75 (m, 2H), 1.20 (d, J=6.6 Hz, 6H), 1.10 (d, J=6.6 Hz, 6H). ¹³C NMR (50 MHz, CDCl₃) δ: 168.5; 142.8; 132.2; 131.3; 129.8; 127.9; 125.2; 51.1; 20.2; 18.3. IR (ATR, cm⁻¹): 3542, 2984, 2940, 1667. HRMS (EI) m/z calculated for C₁₃H₁₉NO₂ ([M+H]⁺): 221.1416. Found: 221.1425.

### 2-(t-butylamino)benzoic acid (10)

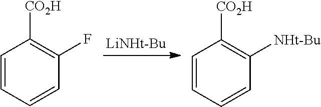

A lithium t-butylamide solution (6 mmol, prepared according to the general procedure in 6 mL of THF) is added dropwise at 0° C. to a 2-fluorobenzoic acid solution 1 (280 mg, 2 mmol) in solution in anhydrous THF (3.4 mL). The reaction mixture is stirred at 0° C. for 72 h before being hydrolyzed by distilled water (30 mL). The pH of the aqueous phase is adjusted to 5 upon addition of an HCl solution (2M) and the solution is extracted with diethyl ether (3*50 mL). The combined organic phases are dried on MgSO₄ and concentrated under reduced pressure. After purification by chromatography on silica gel (eluent=cyclohexane/ethyl acetate 80/20), acid 10 is isolated as a brown solid (140 mg, 36%). Mp=152-153° C. (Coombs, R. V. J. Org. Chem. 1977, 42(10), 1812-1813 151-153° C.). ¹H NMR (400 MHz, CDCl₃) δ: 8.08 (dd, J=1.6 Hz J=8 Hz, 1H, H₆), 7.37 (ddd, J=1.8 Hz J=7.2 Hz J=8.7 Hz, 1H, H₄), 7.19 (d, J=8.3 Hz 1H, H₃), 6.87 (t, J=7.5 Hz, 1H, H₅), 1.40 (s, 9H, (CH₃)₃). ¹³C NMR (50 MHz, CDCl₃) δ: 172.5, 145, 133.3, 132.6, 119.4, 118.3, 117.5, 54.1, 28.6 IR (ATR, cm⁻¹): 2979, 2359, 1676, 1586, 1365, 1199 HRMS. m/z calculated for C₁₁H₁₅NO₂ ([M+H]⁺): 194.1187. Found: 194.1179.

### 2-(diethylamino)-3-methoxybenzoic acid (28)

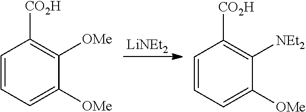

2,3-dimethoxybenzoic acid (364 mg, 2 mmol) in solution in anhydrous THF (4 mL) is added dropwise at 0° C. to a lithium diethylamide solution (10 mmol, prepared according to the general procedure in 8 mL of THF). The solution is stirred at 0° C. for 3 h then hydrolyzed at 0° C. with distilled water (5 mL). The aqueous phase is extracted with ethyl acetate (2*20 mL) and the combined organic phases are washed with an aqueous NaOH solution (10%), dried over MgSO₄ and concentrated under reduced pressure to afford acid 28 as a white solid (237 mg, 53%). The pH of the aqueous phase is adjusted to 7 upon addition of HCl solution (2M) and the aqueous phase is extracted with dichloromethane (3*50 mL). The combined organic phases are dried over MgSO₄ and concentrated under reduced pressure. The raw product obtained is purified by chromatography on silica gel (eluent dichloromethane/methanol:98/2 to 96/4) to afford 88 mg of acid 28. The aqueous phase is then acidified to pH=1 with an aqueous HCl solution (2M) and extracted with ethyl acetate (3*20 mL). The combined organic phases are dried over MgSO₄ and concentrated under reduced pressure. The raw product obtained is purified by chromatography on silica gel (eluent: dichloromethane/methanol: 98/2 to 96/4) to afford 13 mg of acid 28. (overall yield: 338 mg, 74%). Mp: 68-71° C. ¹H NMR (400 MHz, CDCl₃) δ: 7.96 (dd, J=1.4 Hz, J=8.3 Hz, 1H), 7.39 (dd, J=8.0 Hz, J=8.3 Hz, 1H), 7.10 (dd, J=1.4 Hz, J=8.3 Hz, 1H), 3.91 (s, 3H, OCH₃), 3.41 (m, 2H, CH₂), 3.27 (m, 2H, CH₂), 1.06 (t, J=7.4 Hz, 6H, 2*CH₃). ¹³C NMR (100 MHz, CDCl₃) δ: 168.3; 156.0; 131.9; 130.2; 128.8; 123.4; 115.5; 55.8; 48.1; 12.0. IR (ATR, cm⁻¹): 3080, 2980, 1655, 1578, 1476, 1270, 1077, HRMS (EI) m/z calculated for C₁₂H₁₈NO₃ ([M+H]⁺): 224.1287. Found: 224.1281.

### 2-(diethylamino)-3,4-dimethoxybenzoic acid (29)

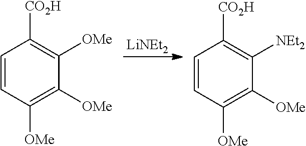

2,3,4-trimethoxybenzoic acid (840 mg, 4 mmol) in solution in anhydrous THF (8 mL) is added dropwise at −30° C. to a lithium diethylamide solution (20 mmol, prepared according to the general procedure in 16 mL of THF). The solution is stirred at −30° C. for 1 h, warm up to 0° C. in 3 h, then hydrolyzed at 0° C. with distilled water (10 mL). The aqueous phase is extracted with ethyl acetate (2*20 mL) and the combined organic phases are washed with an aqueous NaOH solution (10%), then dried over MgSO₄ and concentrated under reduced pressure to afford acid 29 as a white solid (652 mg, 64%). The pH of the aqueous phase is adjusted to 7 upon addition of HCl solution (2M) and the aqueous phase is extracted by dichloromethane (3*30 mL). The combined organic phases are dried over MgSO₄ and concentrated under reduced pressure. The raw product obtained is purified by chromatography on silica gel (eluent: dichloromethane/methanol: 98/2 to 96/4) to afford 119 mg of acid 29. (overall yield: 771 mg, 76%). Mp 57-62° C. ¹H NMR (400 MHz, CDCl₃) δ: 8.08 (d, J=8.9 Hz, 1H), 6.99 (d, J=8.9 Hz, 1H), 3.95 (s, 6H, 2*OCH₃), 3.29 (m, 4H, 2*CH₂), 1.08 (t, J=7.5 Hz, 6H, 2*CH₃). ¹³C NMR (100 MHz, CDCl₃) δ: 168.2; 156.2; 146.0; 137.5; 126.9; 121.5; 111.5; 60.4; 56.0; 48.9; 12.1. IR (ATR, cm⁻¹): 3277, 2976, 2942, 1650, 1591, 1469, 1454, 1270, 1063, 1023, 893. HRMS (EI) m/z calculated for C₁₃H₂₀NO₄ ([M+H]⁺): 254.1392. Found: 254.1360.

### 2-(diethylamino)naphthalene-1-carboxylic acid (32)

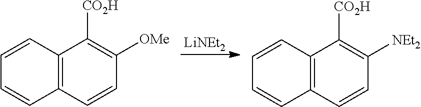

2-methoxynaphthalene-1-carboxylic acid (603 mg, 3 mmol) in solution in anhydrous THF (20 mL) is added dropwise at −78° C. to a lithium diethylamide solution (6.6 mmol, prepared according to the general procedure in 12 mL of THF). The solution is stirred at −78° C. for 2 h, allowed to warm up to room temperature overnight, then is hydrolyzed with distilled water (40 mL). The pH of the aqueous phase is adjusted to 7 upon addition of HCl solution (2M) and the aqueous phase is extracted by dichloromethane (3*50 mL). The combined organic phases are dried over MgSO₄ and concentrated under reduced pressure. The raw product obtained is purified by chromatography on silica gel (eluent: dichloromethane/methanol: 9/2) to afford 73 mg of acid 29 (yield 10%). ¹H NMR (400 MHz, CDCl₃) δ: 10.77 (bs, 1H, CO₂H), 8.98 (d, J=7.1 Hz, 1H), 8.06 (d, J=8.3 Hz, 1H), 7.88 (d, J=8.2 Hz, 1H), 7.36 (d, J=8.3 Hz, 1H), 7.73-7.57 (m, 2H, H-arom), 3.47 (q, J=7.1 Hz, 4H, 2*CH₂), 1.16 (t, J=7.1 Hz, 6H, 2*CH₃). ¹³C NMR (100 MHz, CDCl₃) δ: 151.9; 145.9; 135.3; 129.4; 127.7; 127.4; 126.7; 126.4; 123.6; 118.7; 105.7; 55.3; 14.1. IR (ATR, cm⁻¹): 2963, 1373, 821, 788.

### 1-(diethylamino-naphthalene-2-carboxylic acid (35)

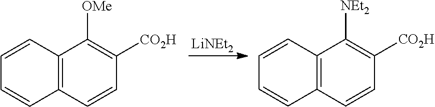

1-methoxynaphthalene-2-carboxylic acid (606 mg, 3 mmol) in solution in anhydrous THF (20 mL) is added dropwise at −78° C. to a lithium diethylamide solution (6.6 mmol, prepared according to the general procedure in 12 mL of THF). The solution is stirred at −78° C. for 2 h, is allowed to warm up to room temperature overnight, then is hydrolyzed with distilled water (40 mL). The pH of the aqueous phase is adjusted to 7 upon addition of HCl solution (2M) and the aqueous phase is extracted by ethyl acetate (3*30 mL). The combined organic phases are dried over MgSO₄ and concentrated under reduced pressure. After recrystallization (Hexane/EtOAc 1/3), acid 35 is isolated as a pale yellow solid (483 mg, 66%). Mp: 95-97° C. ¹H NMR (400 MHz, CDCl₃) δ: 10.49 (bs, 1H, CO₂H), 8.42 (d, J=8.6 Hz, 1H), 8.12 (d, J=7.1 Hz, 1H), 7.98 (d, J=9.6 Hz, 1H), 7.89 (d, J=8.5 Hz, 1H), 7.64-7.57 (m, 2H, H-arom), 3.60 (q, J=7.3 Hz, 4H, 2*CH₂), 1.07 (t, J=7.3 Hz, 6H, 2*CH₃). ¹³C NMR (100 MHz, CDCl₃) δ: 168.3; 142.3; 137.1; 130.0; 128.7; 128.0; 127.4; 127.1; 126.5; 123.7; 118.6; 50.05; 12.7. IR (ATR, cm⁻¹): 3000, 1367, 839, 788. HRMS (EI) m/z calculated for C₁₅H₁₈NO₂ ([M+H]⁺): 244.1339. Found: 244.1338. Microanalysis calculated for C₁₅H₁₇NO₂: C, 74.05; H, 7.04; N, 5.76. Found: C, 73.72; H, 7.03; N, 5.45.

### 2-(N-methyl-N-phenyl)-6-(diethyl)benzoic acid

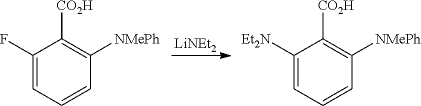

2-(N-methyl-N-phenyl)-6-fluorobenzoic acid (261 mg; 1.1 mmol) in solution in anhydrous THF (10 mL) is added dropwise at −30° C. to a lithium diethylamide solution (5.5 mmol, prepared according to the general procedure in 20 mL of THF). The solution is stirred at −30° C. for 1 h then is allowed to warm up to room temperature overnight. The reaction mixture is hydrolyzed at room temperature with distilled water (20 mL) and the two phases are separated. The aqueous phase (AQ-1) is extracted by ethyl acetate (3*20 mL) and the combined organic phases (ORGA1) are dried over MgSO₄. The ORGA1 phase corresponds predominantly to the carboxylate derived from 2-(N-methyl-N-phenyl)-6-(diethyl)benzoic acid. To purify it, 10 mL of a 1N aqueous NaOH solution and the reaction mixture is concentrated under reduced pressure. After acidification at pH=7 (by HCl 10%) and extraction with AcOEt, pure 2-(N-methyl-N-phenyl)-6-(diethyl)benzoic acid is obtained (200 mg). The aqueous phase AQ-1 is then acidified with an HCl solution (10%) to pH=7 and extracted by dichloromethane (3*20 mL). The combined organic phases (ORGA2) are dried over MgSO₄. After recrystallization of the ORGA2 phase (ethyl acetate/cyclohexane), additional 240 mg of 2-(N-methyl-N-phenyl)-6-(diethyl)benzoic acid are obtained. (overall yield: 320 mg, 98%). Mp=149-150° C. ¹H NMR (CDCl₃; 200 MHz): 7.54 (t; J=8.8 Hz, 1H), 7.34 (dd; J=8.8 Hz; J=1.8 Hz; 1H); 7.22 (d; J=8.8 Hz; J=1.8 Hz; 1H), 7.14 (dd; J=7.2 Hz; J=7.8 Hz; 2H), 6.70 (t; J=7.2 Hz; 1H), 6.60 (d; J=7.8 Hz; 2H), 3.28 (s, 3H), 3.14 (q; J=7.2 Hz; 4H), 1.11 (t; J=7.2 Hz; 6H). ¹³C NMR (CDCl₃; 100 MHz): 165.1, 151.2, 148.9, 133.1, 130.6, 128.8, 119.5, 117.5, 113.9, 51.0, 40.3, 11.7. IR (ATR, cm⁻¹): 2979, 2937, 1592, 1474, 1420, 1380, 1321, 1276, 1229, 1185.

2. Snarab Reaction with Alkyl- and Aryl-Lithium/Magnesium Derivatives

### 1-n-butylnaphthalene-2-carboxylic acid

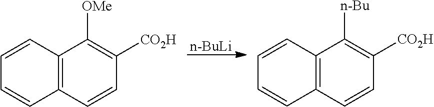

n-BuLi (1.1M in hexane, 6 mL, 6.6 mmol) is added dropwise at −78° C. to a 1-methoxynaphthalene-2-carboxylic acid solution (606 mg, 3 mmol) in 20 ml of anhydrous THF. After 2 h of stirring at −78° C. and then one night at room temperature, the solution is hydrolyzed by distilled water (40 mL), acidified by an HCl solution (2M) and extracted by ethyl acetate (3*30 mL). The combined organic phases are dried over MgSO₄, filtered then concentrated under reduced pressure. After recrystallization (n-hexane/ethyl acetate 1/3), 1-n-butylnaphthalene-2-carboxylic acid is isolated as a pale yellow solid (590 mg, 86%). Mp=98-99° C. (Huisgen, R.; Zirngibl. L Chem. Ber. 1958, 1438.97-97.7° C.). ¹H NMR (400 MHz, CDCl₃) δ: 10.5 (s, 1H), 8.25-8.22 (m, 1H), 7.99 (d, J=8.6 Hz, 1H), 7.87-7.84 (m, 1H), 7.73 (d, J=8.7 Hz, 1H), 7.59-7.55 (m, 2H), 3.49 (t, J=7.5 Hz, 2H), 1.81-1.72 (m, 2H), 1.62-1.53 (m, 2H), 1.05 (t, J=7.2 Hz, 3H). ¹³C NMR (100 MHz, CDCl₃) δ: 174.8, 144.2; 135.6; 132.2; 129; 128.2; 127.7; 126.9; 126.4; 125.9; 125.6; 33.7; 29.2; 23.4; 14. IR (KBr, cm⁻¹): 3000; 1735; 1235; 1069; 982; 768 HRMS m/z calc. for C₁₅H₁₆O₂ ([M+H]⁺): 228.1150 replaced: 228.1159, Microanalysis calc. For C₁₅H₁₆O₂C, 78.92; H, 7.06. Found: C, 78.74; H, 6.99.

### 1-s-butylnaphthalene-2-carboxylic acid

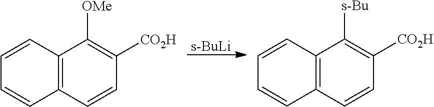

s-BuLi (1.3 M in hexane, 5.1 mL, 6.6 mmol) is added dropwise at −78° C. to a 1-fluoronaphthalene-2-carboxylic acid (570 mg, 3 mmol) in 20 ml of anhydrous THF. After 2 h of stirring at −78° C. and then one night at room temperature, the solution is hydrolyzed with distilled water (40 mL), acidified with an HCl solution (2M) and extracted by ethyl acetate (3*30 mL). The combined organic phases are dried over MgSO₄, filtered then concentrated under reduced pressure. After recrystallization (cyclohexane/ethyl acetate 1/3), 1-s-butylnaphthalene-2-carboxylic acid is isolated as a white solid (590 mg, 86%). Mp=113-114° C. (Mortier, J.; Vaultier, M.; Plunian, B.; Sinbandhit, S. Can. J. Chem. 1999, 77, 98.117-118° C.). ¹H NMR (400 MHz, CDCl₃) δ: 10.7 (s, 1H), 8.4 (m, 1H), 7.9 (m, 1H), 7.75 (m, 2H), 7.55 (m, 2H), 3.9 (m, 1H), 2.1 (m, 2H), 1.65 (d, J=7.2 Hz, 3H), 0.9 (t, J=7 Hz, 3H). ¹³C NMR (100 MHz, CDCl₃) δ: 176.5; 144.5; 135.6; 131.7; 129.6; 129.2; 126.9; 125.9; 125.7; 125.3; 38.5; 29.8; 20.5; 13.3. IR (KBr, cm⁻¹): 2963; 1682; 1279; 1170; 886; 767. HRMS m/z calc. for C₁₅H₁₆O₂ ([M+H]⁺): 228.1150 found 228.1153.

### 1-t-butylnaphthalene-2-carboxylic acid

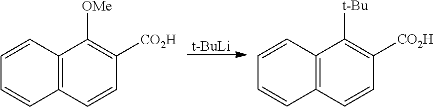

t-BuLi (1.7 M in pentane; 3.9 mL; 6.6 mmol) is added dropwise at −78° C. to a solution of 1-methoxynaphthalene-2-carboxylic acid (606 mg, 3 mmol) in 20 ml of anhydrous THF. After 2 h of stirring at −78° C. and then one night at room temperature, the solution is hydrolyzed by distilled water (40 mL), acidified by an HCl solution (2M) and extracted by ethyl acetate (3*30 mL). The combined organic phases are dried over MgSO₄, filtered then concentrated under reduced pressure. After recrystallization (cyclohexane/ethyl acetate 1/3), 1-t-butyl-2-naphthoic acid is isolated as a white solid (600 mg, 87%). Mp=138-140° C. ¹H NMR (400 MHz, CDCl₃) δ: 10.5 (s, 1H), 8.52 (d, J=7.45 Hz 1H), 7.81 (d, J=7.1 Hz 1H), 7.69 (d, J=8.5 Hz, 1H), 7.52-7.45 (m, 2H), 7.36 (d, J=8.3 Hz, 1H), 1.76 (s, 9H). ¹³C NMR (100 MHz, CDCl₃) δ: 179.9; 143.6; 135.2; 132.2; 130.2; 129.3; 128.3; 127.4; 125.8; 125.6; 125.0; 124.7; 38.1; 32.5. IR (KBr, cm⁻¹): 3000, 1684, 1415, 1037, 938, 774. HRMS m/z calc. for C₁₅H₁₆O₂ ([M+H]⁺): 228.1150 found: 228.1163.

### 1-phenylnaphthalene-2-carboxylic acid

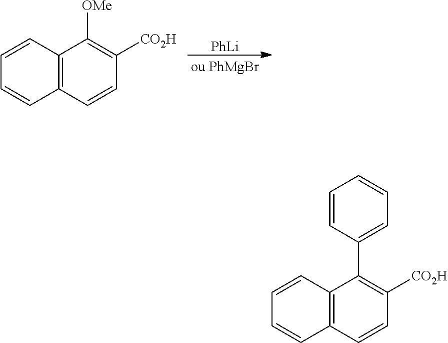

(a) using PhLi as Nucleophile

PhLi (1.0 M in Et₂O; 6.6 mL; 6.6 mmol) is added dropwise at −30° C. to a solution of 1-methoxynaphthalene-2-carboxylic acid (606 mg, 3 mmol) in 20 ml of anhydrous THF. After 2 h of stirring at −30° C. and then one night at room temperature, the solution is hydrolyzed with distilled water (40 mL), acidified with HCl solution (2M) and extracted by ethyl acetate (3*30 mL). The combined organic phases are dried over MgSO₄, filtered then concentrated under reduced pressure. After recrystallization (n-hexane/ethyl acetate 1/3), 1-phenylnaphthalene-2-carboxylic acid is isolated as a pale yellow solid (600 mg, 80%).

(b) Using PhMgBr as Nucleophile

PhMgBr (2.16 M in THF; 3.05 mL, 6.6 mmol) is added dropwise at −30° C. to a solution of 1-methoxynaphthalene-2-carboxylic acid (606 mg, 3 mmol) in 20 ml of anhydrous THF. After 2 h of stirring at −78° C. and then one night at room temperature, the solution is hydrolyzed with distilled water (40 mL), acidified with an HCl solution (2M) and extracted by ethyl acetate (3*30 mL). The combined organic phases are dried over MgSO₄, filtered, then concentrated under reduced pressure. After recrystallization (n-hexane/ethyl acetate 1/3), 1-phenylnaphthalene-2-carboxylic acid is isolated as a pale yellow solid (600 mg, 80%). Mp=145-147° C. (Meyers, A. I.; Lutomski, K. A. Synthesis 1983, 105 147-148.5° C.). ¹H NMR (400 MHz, CDCl₃) δ: 11.1 (s, 1H), 7.91 (d, J=8.5 Hz, 1H), 7.85 (d, J=8.7 Hz, 1H), 7.56-7.48 (m, 2H), 7.43-7.37 (m, 4H), 7.29-7.22 (m, 3H). ¹³C NMR (100 MHz, CDCl₃) δ: 173.8; 142.8; 138.7; 135.2; 132.8; 129.6; 128.1; 128.0; 127.95; 127.8; 127.5; 127.2; 126.7; 126.6; 125.9. IR (KBr, cm⁻¹): 3000; 1692; 1408; 1284; 873; 757. HRMS m/z calc. for C₁₇H₁₂O₂ ([M+H]⁺): 248.0837 found: 228.0869. Microanalysis calc. for C₁₇H₁₂O₂: C, 82.24; H, 4.87. Found: C, 82.03; H, 4.85.

### 2-s-butylnaphthalene-1-carboxylic acid

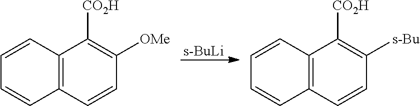

s-BuLi (0.9M in hexane, 7.33 mL, 6.6 mmol) is added dropwise at −78° C. to a solution of 2-methoxynaphthalene-1-carboxylic acid (606 mg, 3 mmol) in 20 ml of anhydrous THF. After stirring 2 h at −78° C. and then one night at room temperature, the solution is hydrolyzed with distilled water (40 mL), acidified with HCl solution (2M) and extracted with ethyl acetate (3*30 mL). The combined organic phases are derived over MgSO₄, filtered, and then concentrated under reduced pressure to afford 2-s-butylnaphthalene-1-carboxylic acid as a white solid (650 mg, 95%). Mp=168-170° C. (Mortier, J; Vaultier, M; Plunian, B.; Sinbandhit, S. Can. J. Chem. 1999, 77, 98. 166-168° C.) ¹H NMR (200 MHz, CDCl₃) δ: 10.60 (s, 1H), 7.91 (d, J=8.8 Hz, 1H), 7.81 (d, J=8.8 Hz, 1H), 7.74 (d, J=8.5 Hz, 1H), 7.52-7.46 (m, 1H), 7.43-7.36 (m, 2H), 3.08-2.98 (m, 1H), 1.75-1.61 (m, 2H), 1.27 (d, J=6.8 Hz, 3H), 0.77 (t, J=7.3 Hz, 3H). ¹³C NMR (100 MHz, CDCl₃) δ: 174.8; 141.3; 130.7; 129.3; 128.8; 128.4; 126.9; 125.8; 123.6; 125.3; 122, 4, 38.05; 29.5; 21.1; 11.3. IR (KBr, cm⁻¹): 2850; 1695; 1400; 1253; 900; 780; 751. HRMS m/z calc. for C₁₇H₁₂O₂ ([M+H]⁺): 228.1150 found: 228.1170.

### 2-(t-butyl)naphthalene-1-carboxylic acid

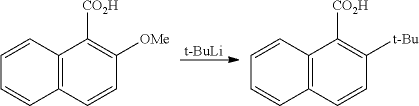

t-BuLi (1.7 M in pentane; 3.9 mL; 6.6 mmol) is added dropwise at −78° C. to a solution of 2-methoxynaphthalene-1-carboxylic acid (606 mg, 3 mmol) in 20 ml of anhydrous THF. After stirring 2 h at −78° C. and then one night at room temperature, the solution is hydrolyzed with distilled water (40 mL), acidified with HCl solution (2M) and extracted with ethyl acetate (3*30 mL). The combined organic phases are dried over MgSO₄, filtered, and then concentrated under reduced pressure. After recrystallization (cyclohexane/ethyl acetate 1/3), 2-t-butyl-1-naphthoic acid is isolated as a white solid (600 mg, 87%). Mp=120-123° C. ¹H NMR (400 MHz, CDCl₃) δ: 10.50 (s, 1H), 7.91 (d, J=8.4 Hz, 1H), 7.85 (d, J=8.8 Hz, 1H), 7.80 (d, J=8.2 Hz, 1H), 7.65 (d, J=8.9 Hz, 1H), 7.57-7.54 (m, 1H), 7.51-7.47 (m, 1H), 1.59 (s, 9H). ¹³C NMR (100 MHz, CDCl₃) δ: 178.7; 143.9; 131.4; 129.9; 129.4; 129.1; 128; 127.8; 126.9; 125.5; 124.5; 36.8; 31.7. IR (KBr, cm⁻¹): 2950; 1685; 1464; 1103; 933; 770; 741. HRMS m/z calc. for C₁₅H₁₆O₂ ([M+H]⁺): 228.1150. Found: 228.1166.

### 1-vinylnaphthalene-2-carboxylic acid

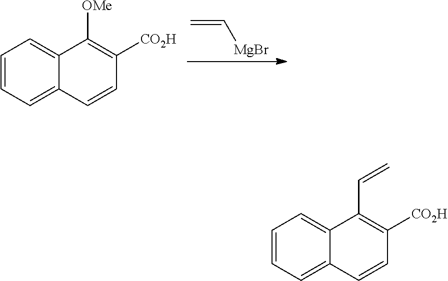

Vinylmagnesium bromide (0.75M in THF; 8.8 mL; 6.6 mmol) is added dropwise to a solution of 1-methoxynaphthalene-2-carboxylic acid (607 mg, 3.0 mmol) in 20 mL of anhydrous THF. The reaction mixture is refluxed two hours, then hydrolyzed at room temperature with distilled water (20 mL), acidified to pH=1 with an aqueous HCl solution (2M) and extracted with ethyl acetate (3*40 mL). The combined organic phases are dried over MgSO₄, filtered then concentrated under reduced pressure. After recrystallization (diethyl ether/petroleum ether), the 1-vinylnaphthalene-2-carboxylic acid is isolated as a white powder (505 mg, 85%). ¹H NMR (400 MHz, CDCl₃) d: 8.38 (d, J=8.8 Hz, 1H), 8.03 (d, J=8.7 Hz, 1H), 7.87 (d, J=8.8 Hz, 1H), 7.83 (d, J=8.7 Hz, 1H), 7.61-7.52 (m, 2H), 7.46 (dd, J=11.5 Hz, J=17.8 Hz, 1H), 5.78 (dd, J=1.8 Hz, J=11.5 Hz, 1H), 5.41 (dd, J=1.8 Hz, J=17.8 Hz, 1H). ¹³C NMR (50 MHz, CDCl₃) d: 173.8; 141.1; 135.7; 134.3; 131.6; 128.1; 128.0; 127.7; 127.3; 126.5; 125.9; 125.1; 120.8. HRMS m/z calculated for C₁₃H₁₀O₂ ([M]⁺): 198.0681 found 198.0680.

### 1-ethylnaphthalene-2-carboxylic acid

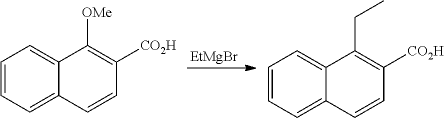

Ethylmagnesium bromide (1.1M in diethyl ether; 6.0 mL; 6.6 mmol) is added dropwise at −78° C. to a solution of 1-methoxynaphthalene-2-carboxylic acid (606 mg, 3.0 mmol) in 20 mL of anhydrous THF. The reaction mixture is stirred two hours at −78° C., then hydrolyzed by distilled water (20 mL), acidified at room temperature to pH=1 with aqueous HCl solution (2M) and extracted with ethyl acetate (3*40 mL). The combined organic phases are dried over MgSO₄, filtered then concentrated under reduced pressure. After recrystallization (n-hexane/ethyl acetate: 1/3), 1-ethylnaphthalene-2-carboxylic acid is isolated as a white solid (560 mg, 93%). Mp=147-149° C. (Jacqueline, G; Bull. Soc. Chim. Fr. 1964, 27. 150° C.). ¹H NMR (400 MHz, acetone-d6) d: 11.71 (s, 1H), 8.25 (d, J=9.0 Hz, 1H), 7.93-7.90 (m, 2H), 7.78 (d, J=8.7 Hz, 1H), 7.62-7.55 (m, 2H), 1.43 (q, J=7.4 Hz, 2H), 1.16 (t, J=7.0 Hz, 3H). ¹³C NMR (100 MHz, acetone-d6) d: 174.4; 148.1; 140.4; 137.0; 133.9; 132.9; 132.4; 132.9; 131.5; 131.4; 130.3; 27.4; 20.5. IR (KBr, cm⁻¹): 3000, 1629, 1450, 1244, 869, 793. HRMS m/z calculated for C₁₃H₁₂O₂ ([M]⁺): 200.0837 found 200.0843.

### 1-(4-methoxyphenyl)naphthalene-2-carboxylic acid

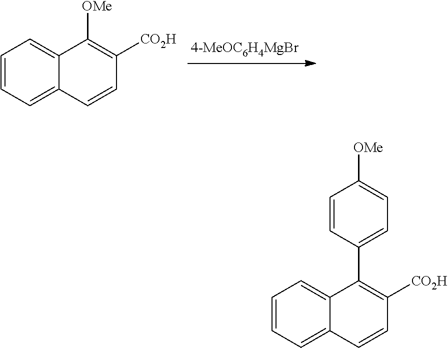

4-methoxyphenylmagnesium bromide (0.85M in THF; 7.8 mL; 6.6 mmol) is added dropwise to a solution of 1-methoxynaphthalene-2-carboxylic acid (607 mg, 3.0 mmol) in 20 mL of anhydrous THF. The reaction mixture is refluxed two hours, then hydrolyzed at room temperature with distilled water (20 mL), acidified to pH=1 with an aqueous HCl solution (2M) and extracted with ethyl acetate (3*40 mL). The combined organic phases are dried over MgSO₄, filtered then concentrated under reduced pressure. After chromatography on silica gel (cyclohexane/ethyl acetate: 9/1 to 0/1), 1-(4-methoxyphenyl)naphthalene-2-carboxylic acid is isolated as a white solid (691 mg, 83%). ¹H NMR (400 MHz, CDCl₃) d: 7.98 (d, J=8.7 Hz, 1H), 7.88 (m, 2H), 7.61 (d, J=8.5 Hz, 1H), 7.57-7.53 (m, 1H), 7.43-7.39 (m, 1H), 7.25-7.21 (m, 2H), 7.02-6.99 (m, 2H), 3.90 (s, 3H). ¹³C NMR (50 MHz, CDCl₃) d: 173.4; 159.0; 142.3; 135.1; 133.0; 130.7; 130.6; 128.0; 127.8; 127.7; 127.6; 126.9; 126.6; 125.8; 113.4; 55.2. HRMS m/z calculated for C₁₈H₁₄O₃ ([M]⁺): 278.0943 found 278.0940.

### 1-(2-methoxyphenyl)naphthalene-2-carboxylic acid

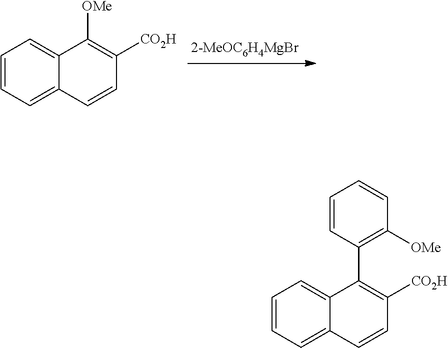

To a solution of 1-methoxynaphthalene-2-carboxylic acid (410 mg, 2.03 mmol) in 15 mL of anhydrous THF is added dropwise ethylmagnesium bromide (2.5M in THF, 0.73 mL, 1.83 mmol) and one hour later 2-methoxyphenylmagnesium bromide (0.27M in THF; 11.3 mL; 3.05 mmol). The reaction mixture is refluxed two hours, then hydrolyzed at room temperature with distilled water (15 mL), acidified to pH=1 with aqueous HCl solution (2M) and extracted with ethyl acetate (3*40 mL). The combined organic phases are dried over MgSO₄, filtered, and then concentrated under reduced pressure. After recrystallization (cyclohexane), 1-(2-methoxyphenyl)naphthalene-2-carboxylic acid is isolated as a white solid (504 mg, 89%). Mp=182-184° C. ¹H NMR (400 MHz, acetone-d6) d: 8.03-7.98 (m, 3H), 7.60-7.56 (m, 1H), 7.50-7.40 (m, 3H), 7.13-7.11 (m, 2H), 7.07-7.03 (m, 1H), 3.63 (s, 3H). ¹³C NMR (100 MHz, acetone-d6) d: 169.0; 158.3; 139.3; 135.8; 133.6; 131.7; 129.8 (2×); 129.0; 128.8; 128.3; 128.2; 128.1; 127.3; 126.8; 121.0; 111.9; 55.8. IR (ATR, cm⁻¹): 2835, 1687, 1492, 1284, 910, 787, 756. HRMS m/z calculated for C₁₈H₁₄O₃ ([M]⁺): 278.0943 found 278.0956.

### 1-(2-methylphenyl)-naphthalene-2-carboxylic acid

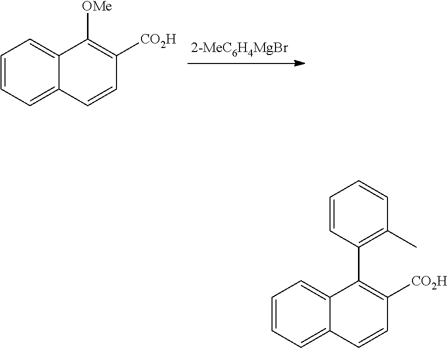

2-methylphenylmagnesium bromide (0.66M in THF; 10.0 mL; 6.6 mmol) is added dropwise to solution of a 1-methoxynaphthalene-2-carboxylic acid (606 mg, 3.0 mmol) in 20 mL of anhydrous THF. The reaction mixture is refluxed two hours, hydrolyzed at room temperature with distilled water (20 mL), acidified to pH=1 with an aqueous HC 1 solution (2M) and extracted with ethyl acetate (3*40 mL). The combined organic phases are dried over MgSO₄, filtered and then concentrated under reduced pressure. After recrystallization (cyclohexane), 1-(2-methylphenyl)naphthalene-2-carboxylic acid is isolated as a white solid (640 mg, 81%). Mp=136-138° C. ¹H NMR (200 MHz, CDCl₃) d: 10.91 (sl, 1H), 8.04 (d, J=8.6 Hz, 1H), 7.87 (d, J=8.9 Hz, 2H), 7.53-7.49 (m, 1H), 7.35-7.28 (m, 3H), 7.27-7.21 (m, 2H), 7.04 (d, J=7.4 Hz, 1H), 1.90 (s, 3H). ¹³C NMR (100 MHz, CDCl₃) d: 172.9; 142.7; 138.4; 136.6; 135.3; 132.6; 129.5; 129.2; 128.0; 127.8; 127.7; 126.8; 126.3; 126.1; 125.5; 124.9; 124.7; 19.9. IR (KBr, cm⁻¹): 2859, 1693, 1464, 1253, 942, 770, 755. HRMS m/z calculated for C₁₈H₁₄O₂ ([M]⁺): 262.0994 found 262.0997.

### 1-(2,5-dimethylphenyl)-naphthalene-2-carboxylic acid

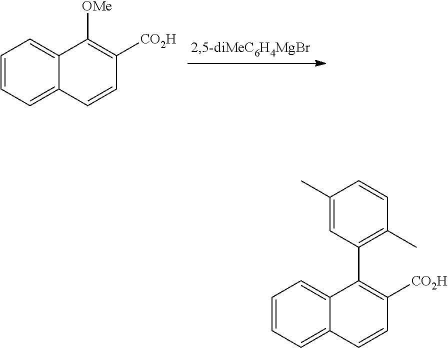

2,5-dimethylphenylmagnesium bromide (0.50M in THF; 13.2 mL; 6.6 mmol) is added dropwise to a solution of 1-methoxynaphthalene-2-carboxylic acid (606 mg, 3.0 mmol) in 20 mL of anhydrous THF. The reaction mixture is refluxed two hours and then hydrolyzed at room temperature with distilled water (20 mL), acidified to pH=1 with an aqueous HCl solution (2M) and extracted with ethyl acetate (3*40 mL). The combined organic phases are dried over MgSO₄, filtered and then concentrated under reduced pressure. After recrystallization (cyclohexane), 1-(2,5-dimethylphenyl)naphthalene-2-carboxylic acid is isolated as a white solid (600 mg, 72%). Mp=165-167° C. ¹H NMR (400 MHz, CDCl₃) d: 8.04 (d, J=8.7 Hz, 1H), 7.87 (d, J=8.7 Hz, 2H), 7.55-7.51 (m, 1H), 7.37 (m, 2H), 7.22-7.13 (m, 2H), 6.89 (s, 1H), 2.32 (s, 3H), 1.88 (s, 3H). ¹³C NMR (100 MHz, CDCl₃) d: 172.8; 142.8; 138.1; 135.4; 134.8; 133.5; 132.6; 129.9; 129.4; 128.4; 128.1; 127.9; 127.8; 127.5; 126.7; 126.3; 126.1; 21.0; 19.3. IR (KBr, cm⁻¹): 2916, 1673, 1410, 1279, 913, 771, 758. HRMS m/z calculated for C₁₉H₁₇O₂ ([M+H]⁺): 277.1229 found 277.1234.

### 1-naphthyl-naphthalene-2-carboxylic acid

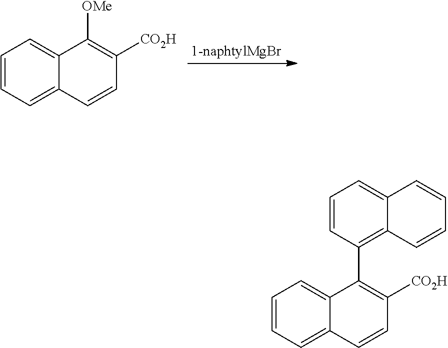

Naphthylmagnesium bromide (0.66M in THF; 10.0 mL; 6.6 mmol) is added dropwise to a solution of 1-methoxynaphthalene-2-carboxylic acid (606 mg, 3.0 mmol) in 20 mL of anhydrous THF. The reaction mixture is refluxed two hours, and then hydrolyzed at room temperature with distilled water (20 mL), acidified to pH=1 with an aqueous HCl solution (2M) and extracted with ethyl acetate (3*40 mL). The combined organic phases are dried over MgSO₄, filtered then concentrated under reduced pressure. After recrystallization (cyclohexane), and then chromatography on silica gel (cyclohexane/ethyl acetate: 3/2), 1-naphthyl-naphthalene-2-carboxylic acid is isolated as a white solid (630 mg, 70%). Mp=180-182° C. (Shindo, M.; Yamamoto, Y.; Yamada, K.; Tomioka, K.; Chem. Pharm. Bull. 2009, 57, 752. 177-184° C.). ¹H NMR (400 MHz, CDCl₃) d: 8.05 (d, J=8.7 Hz, 1H), 7.95-7.89 (m, 4H), 7.54-7.49 (m, 2H), 7.45-7.41 (m, 1H), 7.30-7.20 (m, 4H), 7.12 (d, J=8.4 Hz, 1H). ¹³C NMR (100 MHz, CDCl₃) δ: 172.3; 141.3; 136.5; 135.2; 133.3; 133.2; 132.9; 128.3; 128.2; 128.1; 128.0; 127.9; 127.8; 127.3; 127.0; 126.7; 126.2; 126.1; 125.9; 125.7; 125.3. IR (ATR, cm⁻¹): 2922, 1691, 1461, 1251, 913, 795.768. HRMS m/z calculated for C₂₁H₁₄O₂ ([M+H]⁺): 299.1072 found 299.1077.

### (2-methoxy-1-naphtyl)-naphtalene-2-carboxylic acid

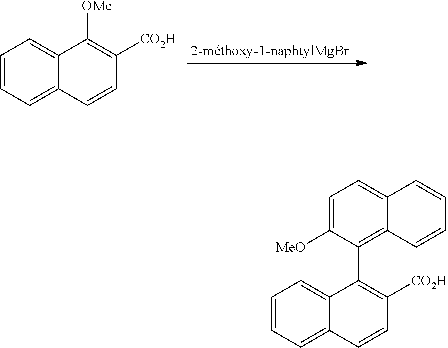

2-methoxy-1-naphthylmagnesium bromide (0.25M in THF; 10.5 mL; 4.4 mmol) is added dropwise to a solution of 1-methoxynaphthalene-2-carboxylic acid (404 mg, 2.0 mmol) in 15 mL of anhydrous THF. The reaction mixture is refluxed two hours then hydrolyzed at room temperature with distilled water (20 mL), acidified to pH=1 with an aqueous HCl solution (2M) and extracted with ethyl acetate (3*40 mL). The combined organic phases are dried over MgSO₄, filtered then concentrated under reduced pressure. After chromatography on silica gel (petroleum ether/ethyl acetate: 9/1 to 0/1) then recrystallization (petroleum ether/ethyl acetate), (2-methoxy-1-naphtyl)-naphtalene-2-carboxylic acid is isolated as a white solid (265 mg, 00%). ¹H NMR (400 MHz, CDCl₃) δ: 8.15 (d, J=8.7 Hz, 1H), 7.99 (d, J=8.8 Hz, 2H), 7.93 (d, J=8.2 Hz, 1H), 7.86 (d, J=8.2 Hz, 1H), 7.53 (ddd, J=1.6 Hz, J=6.4 Hz, J=8.1 Hz, 1H), 7.39 (d, J=9.1 Hz, 1H), 7.32-7.19 (m, 3H), 7.17 (ddd, J=1.3 Hz, J=6.8 Hz, J=8.3 Hz, 1H), 6.90 (d, J=8.5 Hz, 1H), 3.70 (s, 3H). ¹³C NMR (100 MHz, DMSO-d6) δ: 168.1; 153.8; 135.6; 134.4; 133.3; 132.2; 130.0; 129.2; 128.4; 128.0; 127.9; 127.6; 127.4; 126.7; 126.6; 126.2; 126.0; 124.2; 123.1; 121.1; 113.9, 56.1. HRMS m/z calculated for C₂₂H₁₆O₃ ([M+NH₄]): 346.1443 found 346.1425.

### 1-n-butyl-naphthalene-2-carboxylic acid

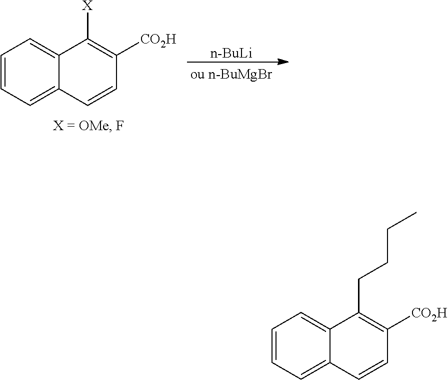

a) Using n-BuLi

n-butyllithium (1.1 M in hexane; 6.0 mL; 6.6 mmol) is added dropwise at −78° C. to a solution of 1-fluoronaphthalene-2-carboxylic acid (570 mg, 3.0 mmol) or 1-methoxynaphthalene-2-carboxylic acid (606 mg, 3.0 mmol) in 20 mL of anhydrous THF. After stirring two hours at −78° C., the reaction mixture is hydrolyzed with distilled water (20 mL), acidified at room temperature to pH=1 with an aqueous HCl solution (2M) and extracted with ethyl acetate (3*40 mL). The combined organic phases are dried over MgSO₄, filtered and concentrated under reduced pressure. After recrystallization (n-hexane/ethyl acetate: 1/3), 1-n-butylnaphthalene-2-carboxylic acid is isolated as a white solid (600 mg, 87% from 1-fluoronaphthalene-2-carboxylic acid; 590 mg, 86% from 1-methoxynaphthalene-2-carboxylic acid).

b) Using n-BuMgBr

n-butylmagnesium bromide (1.0 M in THF; 6.0 mL; 6.6 mmol) is added dropwise at −78° C. to a solution of 1-fluoronaphthalene-2-carboxylic acid (570 mg, 3.0 mmol) in 20 mL of anhydrous THF After stirring two hours at −78° C., the reaction mixture is hydrolyzed with distilled water (20 mL), acidified at room temperature to pH=1 with an aqueous HCl solution (2M) and extracted with ethyl acetate (3*40 mL). The combined organic phases are dried over MgSO₄, filtered then concentrated under reduced pressure. After recrystallization (n-hexane/ethyl acetate: 1/3), 1-n-butylnaphthalene-2-carboxylic acid is isolated as a white solid (560 mg, 81%).

Mp=98-99° C. (Huisgen, R.; Zirngibl. L Chem. Ber. 1958, 1438.97-97.7° C.). ¹H NMR (400 MHz, CDCl₃) δ: 10.5 (s, 1H), 8.25-8.22 (m, 1H), 7.99 (d, J=8.6 Hz, 1H), 7.87-7.84 (m, 1H), 7.73 (d, J=8.7 Hz, 1H), 7.59-7.55 (m, 2H), 3.49 (t, J=7.5 Hz, 2H), 1.81-1.72 (m, 2H), 1.62-1.53 (m, 2H), 1.05 (t, J=7.2 Hz, 3H). ¹³C NMR (100 MHz, CDCl₃) δ: 174.8, 144.2; 135.6; 132.2; 129; 128.2; 127.7; 126.9; 126.4; 125.9; 125.6; 33.7; 29.2; 23.4; 14. IR (KBr, cm⁻¹): 3000; 1735; 1235; 1069; 982; 768 HRMS m/z calculated for C₁₅H₁₆O₂ ([M+H]⁺): 228.1150 found: 228.1159, Microanalysis calc. for C₁₅H₁₆O₂C: 78.92, H: 7.06. found: C: 78.74, H: 6.99.

### 1-s-butyl-naphthalene-2-carboxylic acid

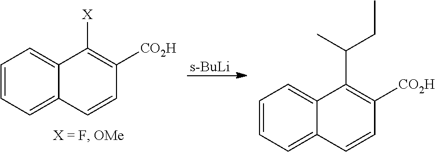

s-butyllithium (1.3 M in hexane; 5.1 mL; 6.6 mmol) is added dropwise at −78° C. to a solution of 1-fluoronaphthalene-2-carboxylic acid (570 mg, 3.0 mmol) or 1-methoxynaphthalene-2-carboxylic acid (606 mg, 3.0 mmol) in 20 mL of anhydrous THF. After stirring two hours at −78° C., the reaction mixture is hydrolyzed with distilled water (20 mL), acidified at room temperature to pH=1 with an aqueous HCl solution (2M) and extracted with ethyl acetate (3*40 mL). The combined organic phases are dried over MgSO₄, filtered then concentrated under reduced pressure. After recrystallization (cyclohexane/ethyl acetate: 1/3), 1-s-butylnaphthalene-2-carboxylic acid is isolated as a white solid (590 mg, 86% from 1-fluoronaphthalene-2-carboxylic acid; 630 mg, 92% from 1-methoxynaphthalene-2-carboxylic acid). Mp=113-114° C. (Mortier, J.; Vaultier, M.; Plunian, B.; Sinbandhit, S. Can. J. Chem. 1999, 77, 98.117-118° C.). ¹H NMR (400 MHz, CDCl₃) δ: 10.7 (s, 1H), 8.4 (m, 1H), 7.9 (m, 1H), 7.75 (m, 2H), 7.55 (m, 2H), 3.9 (m, 1H), 2.1 (m, 2H), 1.65 (d, J=7.2 Hz, 3H), 0.9 (t, J=7 Hz, 3H). ¹³C NMR (100 MHz, CDCl₃) δ: 176.5; 144.5; 135.6; 131.7; 129.6; 129.2; 126.9; 125.9; 125.7; 125.3; 38.5; 29.8; 20.5; 13.3. IR (KBr, cm⁻¹): 2963; 1682; 1279; 1170; 886; 767. HRMS m/z calc. for C₁₅H₁₆O₂ ([M+H]⁺) 228.1150 found 228.1153.

### 1-t-butyl-naphthalene-2-carboxylic acid

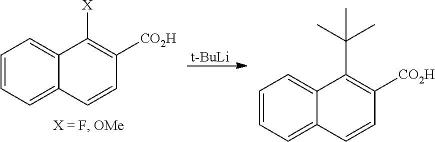

t-butyllithium (1.7 M in pentane; 3.9 mL; 6.6 mmol) is added dropwise at −78° C. to a solution of 1-fluoronaphthalene-2-carboxylic acid (570 mg, 3.0 mmol) or 1-methoxynaphthalene-2-carboxylic acid (606 mg, 3.0 mmol) in 20 mL of anhydrous THF. After stirring two hours at −78° C., the reaction mixture is hydrolyzed with distilled water (20 mL), acidified at room temperature to pH=1 with an aqueous HCl solution (2M) and extracted with ethyl acetate (3*40 mL). The combined organic phases are dried over MgSO₄, filtered and then concentrated under reduced pressure. After recrystallization (cyclohexane/ethyl acetate: 1/3), 1-t-butylnaphthalene-2-carboxylic acid is isolated as a white solid (630 mg, 92% from 1-fluoronaphthalene-2-carboxylic acid; 600 mg, 87% from 1-methoxynaphthalene-2-carboxylic acid). Mp=138-140° C. ¹H NMR (400 MHz, CDCl₃) δ: 10.5 (s, 1H), 8.52 (d, J=7.45 Hz 1H), 7.81 (d, J=7.1 Hz 1H), 7.69 (d, J=8.5 Hz, 1H), 7.52-7.45 (m, 2H), 7.36 (d, J=8.3 Hz, 1H), 1.76 (s, 9H). ¹³C NMR (100 MHz, CDCl₃) δ: 179.9; 143.6; 135.2; 132.2; 130.2; 129.3; 128.3; 127.4; 125.8; 125.6; 125.0; 124.7; 38.1; 32.5. IR (KBr, cm⁻¹): 3000, 1684, 1415, 1037, 938, 774. HRMS m/z calc. for C₁₅H₁₆O₂ ([M+H]⁺) 228.1150 found: 228.1163.

### 1-phenyl-naphthalene-2-carboxylic acid

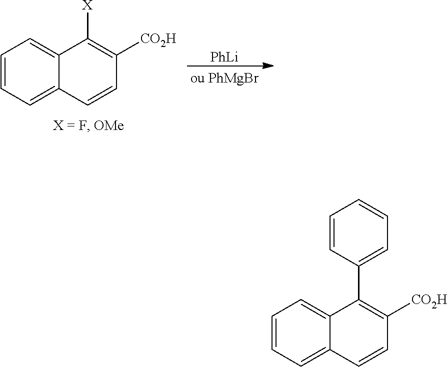

a) Using PhLi

Phenyllithium (1.0 M in di-n-butylether; 6.6 mL; 6.6 mmol) is added dropwise at −30° C. to a solution of 1-fluoronaphthalene-2-carboxylic acid (570 mg, 3.0 mmol) or 1-methoxynaphthalene-2-carboxylic acid solution (606 mg, 3.0 mmol) in 20 mL of anhydrous THF. After stirring two hours at −30° C., the reaction mixture is hydrolyzed with distilled water (20 mL), acidified at room temperature to pH=1 with an aqueous HCl solution (2M) and extracted with ethyl acetate (3*40 mL). The combined organic phases are dried over MgSO₄, filtered and then concentrated under reduced pressure. After recrystallization (n-hexane/ethyl acetate: 1/3), 1-phenyl-2-naphthalene-2-carboxylic acid is isolated as a pale yellow solid (560 mg, 75% from 1-fluoronaphthalene-2-carboxylic acid; 600 mg, 80%)).

b) Using PhMgBr

Phenylmagnesium bromide (2.16 M in THF; 3.05 mL; 6.6 mmol) is added dropwise at −78° C. to a solution of 1-fluoronaphthalene-2-carboxylic acid (570 mg, 3.0 mmol) or 1-methoxynaphthalene-2-carboxylic acid (606 mg, 3.0 mmol) in 20 mL of anhydrous THF. After stirring two hours at −78° C. and then one night at room temperature, the reaction mixture is hydrolyzed with distilled water (20 mL), acidified at room temperature to pH=1 with an aqueous HCl solution (2M) and extracted with ethyl acetate (3*40 mL). The combined organic phases are dried over MgSO₄, filtered then concentrated under reduced pressure. After recrystallization (n-hexane/ethyl acetate: 1/3), 1-phenyl-naphthalene-2-carboxylic acid is isolated as a pale yellow solid (600 mg, 80% from 1-fluoronaphthalene-2-carboxylic acid; 600 mg, 80% from 1-methoxynaphthalene-2-carboxylic acid).

Mp=145-147° C. (Meyers, A. I.; Lutomski, K. A. Synthesis 1983, 105 147-148.5° C.). ¹H NMR (400 MHz, CDCl₃) δ: 11.1 (s, 1H), 7.91 (d, J=8.5 Hz, 1H), 7.85 (d, J=8.7 Hz, 1H), 7.56-7.48 (m, 2H), 7.43-7.37 (m, 4H), 7.29-7.22 (m, 3H). ¹³C NMR (100 MHz, CDCl₃) δ: 173.8; 142.8; 138.7; 135.2; 132.8; 129.6; 128.1; 128.0; 127.95; 127.8; 127.5; 127.2; 126.7; 126.6; 125.9. IR (KBr, cm⁻¹): 3000; 1692; 1408; 1284; 873; 757. HRMS m/z calc. for C₁₇H₁₂O₂ ([M+H]⁺) 248.0837 found: 228.0869. Microanalysis calc. for C₁₇H₁₂O₂: C: 82.24, H: 4, 87. found: C: 82.03, H: 4.85.

### 2-phenyl-naphthalene-1-carboxylic acid

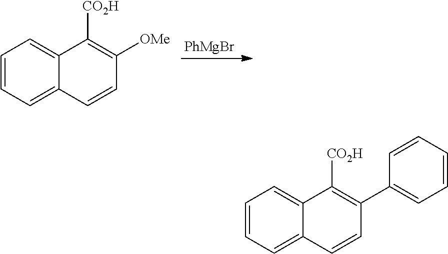

Phenylmagnesium bromide (0.20 M in THF; 33.0 mL; 6.6 mmol) is added dropwise to a solution of 2-methoxynaphthalene-1-carboxylic acid (606 mg, 3.0 mmol) in 20 mL of anhydrous THF. The reaction mixture is refluxed for two hours, and then hydrolyzed at room temperature with distilled water (20 mL), acidified to pH=1 with an aqueous HCl solution (2M) and extracted with ethyl acetate (3*40 mL). The combined organic phases are dried over MgSO₄, filtered then concentrated under reduced pressure. After recrystallization (cyclohexane/ethyl acetate: 1/3), 2-phenyl-naphthalene-1-carboxylic acid is isolated as a white solid (506 mg, 68%). Mp=118-120° C. (Alaka, R.; Indian J. Chem. 1967, 5, 610. 114° C.). ¹H NMR (400 MHz, DMSO-d6) d: 8.29 (d, J=7.8 Hz, 1H), 7.88-7.83 (m, 2H), 7.73 (d, J=6.6 Hz, 2H), 7.47-7.44 (m, 2H), 7.33-7.25 (m, 4H). IR (ATR, cm⁻¹): 3049, 1693, 1463, 1333, 861, 759. HRMS m/z calculated for C₁₇H₁₃O₂ ([M+H]⁺): 249.0916 found 249.0940.

1. Process for preparing aromatic carboxylic acid derivatives by nucleophilic aromatic substitution, in which the following are reacted:
an aromatic carboxylic acid derivative bearing carboxyl function and a single one, or one of the salts thereof, said carboxylic acid derivative has, in the ortho position of the carboxyl function, a leaving group, which is preferably a fluorine or chlorine atom or a chiral or non-chiral alkoxy group, and in this last case, a methoxy group is preferred;
said carboxylic acid derivative being not substituted:
by another electron withdrawing group than the leaving group if any,
by a phenyl group, substituted in para position, especially by a benzyloxy in para position, when the leaving group is a fluorine or chlorine atom;
with a MNu reactant, in which M is a metal and Nu is a chiral or non-chiral nucleophile,
said nucleophilic aromatic substitution reaction being performed without catalyst and without a step of protection/deprotection of the acid function of starting compound.

2. Process according to claim 1, characterized in that said aromatic carboxylic acid derivative, starting compound of the reaction, is a benzoic acid derivative of general formula 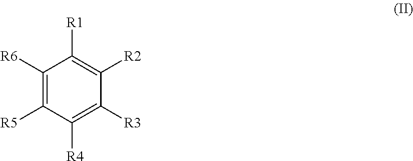
in which
R1 is CO₂H, and R2 is a fluorine or chlorine atom or an alkoxy group, chiral or not, preferably OCH₃; or
R1 is a fluorine or chlorine atom or an alkoxy group, chiral or not, preferably OCH₃ and R2 is CO₂H
R3 is a hydrogen atom, an alkyl group, an alkoxy group, an aryl or an amine substituted or not by one or two alkyl groups, or R3 forms with R4 an aromatic ring or not, or a heterocycle, optionally substituted, in particular by a functional group; or
is a substituent capable of reacting in presence of a base and a metal to form MNu;
R4 is a hydrogen atom, an alkyl group, an alkoxy group, preferably OCH₃, an aryl or an amine substituted or not by one or two alkyl groups, or R4 forms with R3 an aromatic ring or not, or a heterocycle, optionally substituted, in particular by a functional group, or R4 forms with R5 an aromatic ring or not, or a heterocycle, optionally substituted, in particular by a functional group; or is a substituent capable of reacting in presence of a base and a metal to form MNu;
R5 is a hydrogen atom, an alkyl group, an alkoxy group, an aryl or an amine substituted or not by one or two alkyl groups or R5 forms with R4 an aromatic ring or not, or a heterocycle, optionally substituted, in particular by a functional group, or R5 forms with R6 an aromatic ring or not, or a heterocycle, optionally substituted, in particular by a functional group; or is a substituent capable of reacting in presence of a base and a metal to form MNu;
R6 is a hydrogen atom, an alkyl group, an alkoxy group, an aryl or an amine substituted or not by one or two alkyl groups, or R6 forms with R5 and aromatic ring or not, or a heterocycle, optionally substituted, in particular by a functional group; or is a substituent capable of reacting in presence of a base and a metal to form MNu;
which reacts with
a compound (III) of general formula NuM in which Nu is a nucleophile, and M is a metal, preferably Li, Mg, Zn, Cu or an organomagnesium derivative MgX in which X is a halogen atom or an alkoxy group, chiral or not, preferably OCH₃,
said nucleophilic aromatic substitution reaction being performed without catalyst and without step of protection/deprotection of the acid function of the compound (II),
in order to obtain a compound of general formula (I), which corresponds to the general formula (II) in which the one of R1 or R2 that is not CO₂H has been substituted by Nu.

3. Process according to claim 2, in which R1 is CO₂H, R2 is a halogen atom, preferably fluorine or an alkoxy group, chiral or not, preferably methoxy, and R3 to R6 are preferably each a hydrogen atom.

4. Process according to claim 1, in which R1 is CO₂H, R2 is a halogen atom, preferably fluorine or an alkoxy group, chiral or not, preferably methoxy, R3 and R4, or R4 and R5, or R5 and R6 form together a ring, optionally substituted, such that the starting aromatic carboxylic acid derivative is a naphthalene derivative of general formulae (IIa, IIb or IIc) below, in which R7, R8, R9 and R10 are each independently a hydrogen atom, an alkyl group, an alkoxy group, an aryl or an amine substituted or not by one or two alkyl groups; and substituents R3, R4, R5 and R6 not member of the ring are as defined above

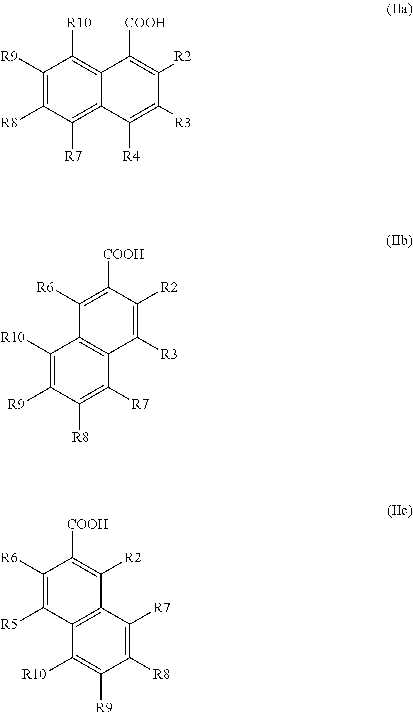

5. Process according to claim 1, in which compound NuM is obtained by reaction of the nucleophile and n-BuLi.

6. Process according to claim 1, in which an asymmetric carbon is present on a leaving group of said aromatic acid derivative, starting compound of the reaction, and/or on the nucleophile, and the compound of general formula (I) obtained is asymmetric.

7. Process according to claim 1, in which NuM is such that M is Li, Mg, Cu, Zn, or MgX in which X is a halogen or an alkoxy, and Nu is as described below:
Nu
Alkyl, preferably CH₃ or C₂H₅
Alkenyl, optionally
substituted
Alkynyl optionally
substituted
Aryl optionally substituted
s-Bu
t-Bu
n-Bu
4-MeOC₆H₄
2-MeOC₆H₄
2,5-diMeC₆H₄
4-Me₂NC₆H₄ 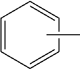
2-MeC₆H₄ 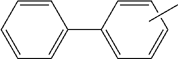 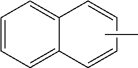 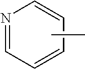 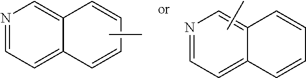 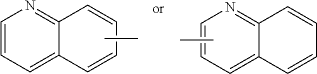 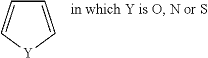 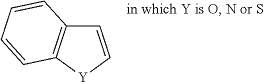
P(Aryl)₂
PArylAlkyl
O(C₁₋₆alkyl)
S(C₁₋₆alkyl) 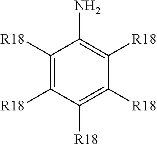
in which R18 is a hydrogen atom, an alkyl group, an alkoxy group, an aryl or an amine substituted or not by one or two C₁₋₁₂alkyl groups, with the condition that the reaction does not involve LiHMDS as base.

8. Process according to claim 1, in which NuM is such that M is Li, Mg, Cu, Zn, or MgX in which X is a halogen or an alkoxy and Nu is N(C₁₋₆alkyl)₂, NH(C₁₋₆alkyl), NEt₂, N(CH₂CH₂)₂NMe, NMeBn, NBn₂, NMePh, NHt-Bu or NPh₂.

9. Process according to claim 1, in which NuM is such that M is Li, Mg, Cu, Zn, or MgX in which X is a halogen or an alkoxy, and Nu is as described below:
Nu
N(C₁₋₆alkyl)₂
NH(C₁₋₆alkyl), in
particular NH(tBu)
NEt₂ 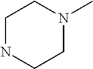
N(iPr)₂ 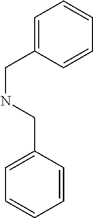 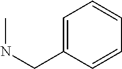 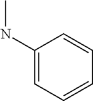
N(CH₂CH₂)₂NMe
NMeBn
NBn₂
NMePh
NHt-Bu
NPh₂

10. Process according to claim 1, in which NuM is such that M is Li, Mg, and Nu is as described below:
Nu 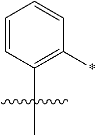 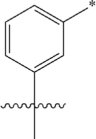 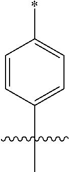 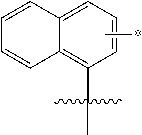 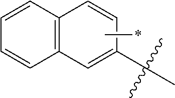 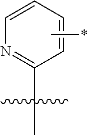 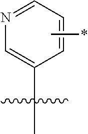 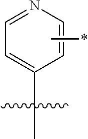 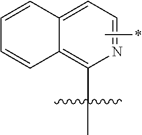 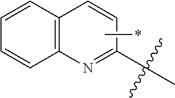 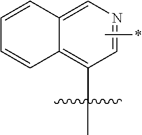 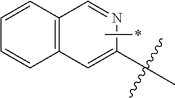 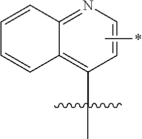 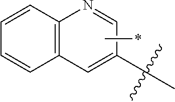 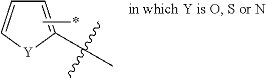 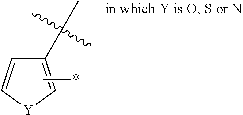 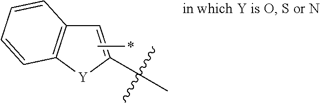 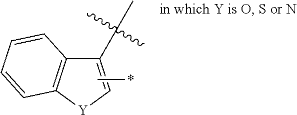
NR¹¹R¹²* in which R¹¹ and R¹² are
each independently a hydrogen
atom, an alkyl group, an alkoxy
group, an aryl, or an amine
substituted or not by one or two
C₁₋₁₂alkyl groups.
SiR¹³R¹⁴R¹⁵* in which R¹³, R¹⁴ and
R¹⁵ are each independently a
hydrogen atom, an alkyl group, an
alkoxy group, an aryl, or an
amine substituted or not by one
or two C₁₋₁₂alkyl groups.
OR¹⁶* in which R¹⁶ is a hydrogen
atom, an alkyl group, an alkoxy
group, an aryl, or an amine
substituted or not by one or two
C₁₋₁₂alkyl groups.
SR¹⁷* in which R¹⁷ is a hydrogen
atom, an alkyl group, an alkoxy
group, an aryl, or an amine
substituted or not by one or two
C₁₋₁₂alkyl groups.
*chiral element

11. Process according to claim 1 in which the product of formula (I) is apogossypol, gossypol or a derivative of these compounds, obtained by the reaction of the compound of the following formula (IId) with the following NuM:

| (IId) | NuM |
|---|---|
| 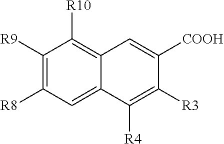 in which R4, R8 and R9 are each independently an alkoxy group and R3 is an alkoxy group with an asymmetric carbon | 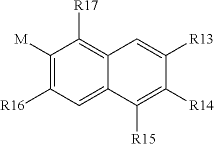 in which R13, R14 and R17 are each independently an alkoxy group and R15 and R16 are each independently an alkyl group |

12. Process according to claim 1, in which the product of formula (I) is benzo[c]phenanthridine, benzo[c][1,7]phenanthroline, benzo[c][1,8]phenanthroline, benzo[c][1,9]phenanthroline, benzo[c][1,10]phenanthroline, pyridazino[4,5-c]phenanthridine.

13. Process according to claim 1, in which at least one equivalent of NuM is used for one equivalent of starting aromatic carboxylic acid derivative.

14. Process according to claim 1, in which at least one equivalent of a metal base, preferably butyllithium, sodium hydride, potassium hydride or lithium hydride is used for one equivalent of starting aromatic carboxylic acid derivative in order to form the metallic salt corresponding to the acid function of the aromatic carboxylic acid derivative, and at least one equivalent of NuM is added for each leaving group of the starting molecule to be substituted.
